(19) United States
(12) Patent Application Publication
(10) Pub. No.: US 2010/0113581 A1
(43) Pub. Date: May 6, 2010

(54) COMBINATION THERAPIES FOR THE TREATMENT OF OBESITY

(76) Inventor: Louis J. Aronne, Greenwich, CT (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/580,447

(22) Filed: Oct. 16, 2009

Related U.S. Application Data
(60) Provisional application No. 61/105,937, filed on Oct. 16, 2008.
(60) Provisional application No. 61/162,022, filed on Mar. 20, 2009.

(51) Int. Cl. A61K 31/365; A61P 3/04

(52) U.S. Cl. 514/449

(57) ABSTRACT

Described are pharmaceutical compositions comprising diethylpropion, metformin, orlistat, and at least one pharmaceutically acceptable carrier or excipient. Another aspect of the present invention relates to a method of treating a patient suffering from obesity or needing to lose weight, comprising the step of co-administering to said patient a therapeutically effective amount of diethylpropion, metformin, and orlistat. In certain embodiments, an aforementioned method is practiced in conjunction or tandem with a medical procedure or the use of a medical device or both.

### RELATED APPLICATIONS

This application claims the benefit of priority to U.S. Provisional Patent Application Ser. Nos. 61/105,937, filed Oct. 16, 2008; and 61/162,022, filed Mar. 20, 2009.

### BACKGROUND OF THE INVENTION

About 100 million adults in the United States are overweight or obese. The medical problems caused by overweight and obesity can be serious and often life-threatening, and include diabetes, shortness of breath, gallbladder disease, hypertension, elevated blood cholesterol levels, cancer, arthritis, other orthopedic problems, reflux esophagitis (heartburn), snoring, sleep apnea, menstrual irregularities, infertility and heart trouble. Moreover, obesity and overweight substantially increase the risk of morbidity from hypertension, dyslipidemia, type 2 diabetes, coronary heart disease, stroke, gallbladder disease, osteoarthritis and endometrial, breast, prostate, and colon cancers. Higher body weights are also associated with increases in all-cause mortality. Most or all of these problems are relieved or improved by permanent significant weight loss. Longevity is likewise significantly increased by permanent significant weight loss.

Prior to 1994, obesity was generally considered a psychological problem. The discovery of the adipostatic hormone leptin in 1994 (Zhang et al., “Positional cloning of the mouse obese gene and its human homologue,” Nature 1994; 372:425-432) brought forth the realization that, in certain cases, obesity may have a biochemical basis. A corollary to this realization was the idea that the treatment of obesity may be achieved by chemical approaches.

Since then, weight loss treatments have varied depending, at least in part, on the degree of weight loss one is attempting to achieve in a subject as well as on the severity of overweight or obesity exhibited by the subject. For example, treatments such as low-fat diet or regular exercise are often adequate in cases where a subject is only mildly overweight. Such treatments can be enhanced by controlled use of over-the-counter appetite suppressants including caffeine, ephedrine and phenylpropanolamine. Moreover, prescription medications including amphetamine, diethylpropion, mazindol, orlistat, phenmetrazine, phendimetrazine, benzphetamine, and fluoxetine are often used in the treatment of seriously overweight or obese subjects or patients. However, such treatments, at best, result in only about 5% to about 10% weight loss (when accompanied with diet and exercise). Moreover, most of these treatments ultimately prove inadequate because they are either dangerous, ineffective, or quickly lose their anorexient effect.

In general, available weight loss drugs have limited efficacy and some clinically significant side effects. Studies of the weight loss medications dexfenfluramine (Guy-Grand, B. et al. (1989) Lancet 2:1142 5), orlistat (Davidson, M. H. et al. (1999) JAMA 281:235 42), sibutramine (Bray, G. A. et al. (1999) Obes. Res. 7:189 98), and orlistat (Douglas, A. et al. (1983) Int. J. Obes. 7:591 5) have shown similar effectiveness. Studies for each demonstrated a weight loss of about 5% of body weight for drug compared with placebo. Other serious considerations limit the clinical use of these drugs. Dexfenfluramine was withdrawn from the market because of suspected heart valvulopathy, orlistat is limited by GI side effects, sibutramine can cause hypertension, and orlistat has limited efficacy.

There have been few combination chemical treatments for obesity. The most famous of these attempts was the introduction of Fen-Phen, a combination of fenfluramine and orlistat. Unfortunately, it was discovered that fenfluramine caused heart-valve complications, which in some cases resulted in the death of the user. Fenfluramine has since been withdrawn from the market. There has been some limited success with other combination therapy approaches, particularly in the field of psychological eating disorders. One such example is Devlin, et al., Int. J. Eating Disord. 28:325-332, 2000, in which a combination of orlistat and fluoxetine showed some efficacy in the treatment of binge eating disorders. Of course, this disorder is an issue for only a small portion of the population.

Accordingly, there exists a need for new, more effective weight loss treatments which are accompanied by fewer adverse or undesirable side effects or less serious side effects. In particular, there exists a need for developing medical weight loss treatments which can potentially lower major endpoints such as death or myocardial infarction rates by directly treating obesity rather than treating the consequences of obesity (e.g., diabetes, hypertension, hyperlipidemia), as is currently the practice.

### SUMMARY OF THE INVENTION

The present invention relates generally to pharmaceutical compositions, and methods of use thereof, containing two or more active agents that, when taken together, result in weight loss for a patient. In certain embodiments, the present invention relates to a pharmaceutical composition comprising diethylpropion, metformin, orlistat, and at least one pharmaceutically acceptable carrier or excipient. In certain embodiments, the present invention relates to a pharmaceutical composition consisting essentially of diethylpropion, metformin, orlistat, and at least one pharmaceutically acceptable carrier or excipient. In certain embodiments, the present invention relates to a pharmaceutical composition consisting of diethylpropion, metformin, orlistat, and at least one pharmaceutically acceptable carrier or excipient.

Another aspect of the present invention relates to a method of treating a patient suffering from obesity, comprising the step of co-administering to said patient a therapeutically effective amount of diethylpropion, metformin, and orlistat. Yet another aspect of the present invention relates to a method of achieving weight loss in a patient, comprising the step of co-administering to said patient a therapeutically effective amount of diethylpropion, metformin, and orlistat. In certain embodiments, an aforementioned method is practiced in conjunction or tandem with a medical procedure or the use of a medical device or both designed to contribute to the overall course of treatment.

### BRIEF DESCRIPTION OF THE FIGURE

FIG. 1 tabulates data gathered from a patient administered a combination of diethylpropion, metformin, and orlistat.

### DETAILED DESCRIPTION OF THE INVENTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used to mean “including but not limited to.” “Including” and “including but not limited to” are used interchangeably.

The term “patient” refers to a mammal in need of a particular treatment. In certain embodiments, a patient is a primate, canine, feline, or equine. In certain embodiments, a patient is a human.

The terms “co-administration” and “co-administering” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

The term “solvate” refers to a pharmaceutically acceptable form of a specified compound, with one or more solvent molecules, that retains the biological effectiveness of such compound. Examples of solvates include compounds of the invention in combination with solvents such, for example, water (to form the hydrate), isopropanol, ethanol, methanol, dimethyl sulfoxide, ethyl acetate, acetic acid, ethanolamine, or acetone. Also included are formulations of solvate mixtures such as a compound of the invention in combination with two or more solvents.

The term “body mass index” (BMI) is used to mean a statistical measurement which compares a person's weight and height. Though it does not actually measure the percentage of body fat, it is a useful tool to estimate a healthy body weight based on how tall a person is. Body mass index is defined as the individual's body weight divided by the square of their height. The formulas universally used in medicine produce a unit of measure of kg/m².

The term “obesity” is used to mean a condition in which excess body fat has accumulated to such an extent that health may be negatively affected. It is commonly defined as a BMI of about 30 kg/m² or higher. This distinguishes it from being “overweight,” as defined by a BMI of between about 25-29.9 kg/m².

### Combination Therapy

One aspect of the present invention relates to combination therapy. This type of therapy is advantageous because the co-administration of active ingredients achieves a therapeutic effect that is greater than the therapeutic effect achieved by administration of only a single therapeutic agent.

In certain embodiments, the co-administration of two or more therapeutic agents achieves a therapeutic effect that is greater than the therapeutic effect achieved by administration of only a single therapeutic agent. In this regard, the combination therapies are efficacious. The therapeutic effect of one therapeutic agent is augmented by the co-administration of another therapeutic agent.

In certain embodiments, the co-administration of two or more therapeutic agents achieves a therapeutic effect that is equal to about the sum of the therapeutic effects achieved by administration of each single therapeutic agent. In these embodiments, the combination therapies are said to be “additive.”

In certain embodiments, the co-administration of two or more therapeutic agents achieves a synergistic effect, i.e., a therapeutic effect that is greater than the sum of the therapeutic effects of the individual components of the combination.

The active ingredients that comprise a combination therapy may be administered together via a single dosage form or by separate administration of each active agent. In certain embodiments, the first and second therapeutic agents are administered in a single dosage form. In certain embodiments, the first, second, and third therapeutic agents are administered in a single dosage form. The agents may be formulated into a single tablet, pill, capsule, or solution for parenteral administration and the like.

In certain embodiments, the therapeutic agents are administered in a single dosage form, wherein each individual therapeutic agent is isolated from the other therapeutic agent(s). Formulating the dosage forms in such a way assists in maintaining the structural integrity of potentially reactive therapeutic agents until they are administered. A formulation of this type may be useful during production and for long-term storage of the dosage form. In certain embodiments, the therapeutic agents may comprise segregated regions or distinct caplets or the like housed within a capsule. In certain embodiments, the therapeutic agents are provided in isolated layers comprised by a tablet.

Alternatively, the therapeutic agents may be administered as separate compositions, e.g., as separate tablets or solutions. One or more active agent may be administered at the same time as the other active agent(s) or the active agents may be administered intermittently. The length of time between administrations of the therapeutic agents may be adjusted to achieve the desired therapeutic effect. In certain instances, one or more therapeutic agent(s) may be administered only a few minutes (e.g., about 1, 2, 5, 10, 30, or 60 min) after administration of the other therapeutic agent(s). Alternatively, one or more therapeutic agent(s) may be administered several hours (e.g., about 2, 4, 6, 10, 12, 24, or 36 hr) after administration of the other therapeutic agent(s). In certain embodiments, it may be advantageous to administer more than one dosage of one or more therapeutic agent(s) between administrations of the remaining therapeutic agent(s). For example, one therapeutic agent may be administered at 2 hours and then again at 10 hours following administration of the other therapeutic agent(s). Importantly, it is required that the therapeutic effects of each active ingredient overlap for at least a portion of the duration of each therapeutic agent so that the overall therapeutic effect of the combination therapy is attributable in part to the combined or synergistic effects of the combination therapy.

The dosage of the active agents will generally be dependent upon a number of factors including pharmacodynamic characteristics of each agent of the combination, mode and route of administration of active agent(s), the health of the patient being treated, the extent of treatment desired, the nature and kind of concurrent therapy, if any, and the frequency of treatment and the nature of the effect desired. In general, dosage ranges of the active agents often range from about 0.001 to about 250 mg/kg body weight per day. For a normal adult having a body weight of about 70 kg, a dosage may range from about 0.1 to about 25 mg/kg body weight. However, some variability in this general dosage range may be required depending upon the age and weight of the subject being treated, the intended route of administration, the particular agent being administered and the like. Since two or more different active agents are being used together in a combination therapy, the potency of each agent and the interactive effects achieved using them together must be considered. Importantly, the determination of dosage ranges and optimal dosages for a particular mammal is also well within the ability of one of ordinary skill in the art having the benefit of the instant disclosure.

In certain embodiments, it may be advantageous for the pharmaceutical combination to have a relatively large amount of the first component compared to the second component. In certain instances, the ratio of the first active agent to second active agent is about 200:1, 190:1, 180:1, 170:1, 160:1, 150:1, 140:1, 130:1, 120:1, 110:1, 100:1, 90:1, 80:1, 70:1, 60:1, 50:1, 40:1, 30:1, 20:1, 15:1, 10:1, 9:1, 8:1, 7:1, 6:1, or 5:1. In certain embodiments, it may be preferable to have a more equal distribution of pharmaceutical agents. In certain instances, the ratio of the first active agent to the second active agent is about 4:1, 3:1, 2:1, 1:1, 1:2, 1:3, or 1:4. In certain embodiments, it may be advantageous for the pharmaceutical combination to have a relatively large amount of the second component compared to the first component. In certain instances, the ratio of the second active agent to the first active agent is about 30:1, 20:1, 15:1, 10:1, 9:1, 8:1, 7:1, 6:1, or 5:1. In certain instances, the ratio of the second active agent to first active agent is about 100:1, 90:1, 80:1, 70:1, 60:1, 50:1, or 40:1. In certain instances, the ratio of the second active agent to first active agent is about 200:1, 190:1, 180:1, 170:1, 160:1, 150:1, 140:1, 130:1, 120:1, or 110:1. Importantly, a composition comprising any of the above-identified combinations of first therapeutic agent and second therapeutic agent may be administered in divided doses about 1, 2, 3, 4, 5, 6, or more times per day or in a form that will provide a rate of release effective to attain the desired results. In one embodiment, the dosage form contains both the first and second active agents. In one embodiment, the dosage form only has to be administered one time per day and the dosage form contains both the first and second active agents.

For example, a formulation intended for oral administration to humans may contain from about 0.1 mg to about 5 g of the first therapeutic agent and about 0.1 mg to about 5 g of the second therapeutic agent, both of which are compounded with an appropriate and convenient amount of carrier material varying from about 5 to about 95 percent of the total composition. Unit dosages will generally contain between about 0.5 mg to about 1500 mg of the first therapeutic agent and 0.5 mg to about 1500 mg of the second therapeutic agent. In certain embodiments, the dosage is about 25 mg, 50 mg, 100 mg, 200 mg, 300 mg, 400 mg, 500 mg, 600 mg, 800 mg, or 1000 mg, etc., up to about 1500 mg of the first therapeutic agent. In certain embodiments, the dosage is about 25 mg, 50 mg, 100 mg, 200 mg, 300 mg, 400 mg, 500 mg, 600 mg, 800 mg, or 1000 mg, etc., up to about 1500 mg of the second therapeutic agent.

In certain embodiments, it may be advantageous for the pharmaceutical combination to have a relatively large amount of the first component compared to the third component. In certain instances, the ratio of the first active agent to third active agent is about 200:1, 190:1, 180:1, 170:1, 160:1, 150:1, 140:1, 130:1, 120:1, 110:1, 100:1, 90:1, 80:1, 70:1, 60:1, 50:1, 40:1, 30:1, 20:1, 15:1, 10:1, 9:1, 8:1, 7:1, 6:1, or 5:1. In certain embodiments, it may be preferable to have a more equal distribution of pharmaceutical agents. In certain instances, the ratio of the first active agent to the third active agent is about 4:1, 3:1, 2:1, 1:1, 1:2, 1:3, or 1:4. In certain embodiments, it may be advantageous for the pharmaceutical combination to have a relatively large amount of the third component compared to the first component. In certain instances, the ratio of the third active agent to the first active agent is about 30:1, 20:1, 15:1, 10:1, 9:1, 8:1, 7:1, 6:1, or 5:1. In certain instances, the ratio of the third active agent to first active agent is about 100:1, 90:1, 80:1, 70:1, 60:1, 50:1, or 40:1. In certain instances, the ratio of the third active agent to first active agent is about 200:1, 190:1, 180:1, 170:1, 160:1, 150:1, 140:1, 130:1, 120:1, or 110:1. Importantly, a composition comprising any of the above-identified combinations of first therapeutic agent and third therapeutic agent may be administered in divided doses about 1, 2, 3, 4, 5, 6, or more times per day or in a form that will provide a rate of release effective to attain the desired results. In certain embodiments, the dosage form contains both the first and third active agents. In certain embodiments, the dosage form only has to be administered one time per day and the dosage form contains both the first and third active agents.

In certain embodiments, it may be advantageous for the pharmaceutical combination to have a relatively large amount of the second component compared to the third component. In certain instances, the ratio of the second active agent to third active agent is about 200:1, 190:1, 180:1, 170:1, 160:1, 150:1, 140:1, 130:1, 120:1, 110:1, 100:1, 90:1, 80:1, 70:1, 60:1, 50:1, 40:1, 30:1, 20:1, 15:1, 10:1, 9:1, 8:1, 7:1, 6:1, or 5:1. In certain embodiments, it may be preferable to have a more equal distribution of pharmaceutical agents. In certain instances, the ratio of the second active agent to the third active agent is about 4:1, 3:1, 2:1, 1:1, 1:2, 1:3, or 1:4. In certain embodiments, it may be advantageous for the pharmaceutical combination to have a relatively large amount of the third component compared to the second component. In certain instances, the ratio of the third active agent to the second active agent is about 30:1, 20:1, 15:1, 10:1, 9:1, 8:1, 7:1, 6:1, or 5:1. In certain instances, the ratio of the third active agent to second active agent is about 100:1, 90:1, 80:1, 70:1, 60:1, 50:1, or 40:1. In certain instances, the ratio of the third active agent to second active agent is about 200:1, 190:1, 180:1, 170:1, 160:1, 150:1, 140:1, 130:1, 120:1, or 110:1. Importantly, a composition comprising any of the above-identified combinations of second therapeutic agent and third therapeutic agent may be administered in divided doses about 1, 2, 3, 4, 5, 6, or more times per day or in a form that will provide a rate of release effective to attain the desired results. In one embodiment, the dosage form contains both the second and third active agents. In another embodiment, the dosage form only has to be administered one time per day and the dosage form contains both the second and third active agents.

In certain embodiments, the dosage form contains the first, the second, and the third active agents. In certain embodiments, the dosage form only has to be administered one time per day and the dosage form contains the first, the second, and the third active agents.

For example, a formulation intended for oral administration to humans may contain from about 0.1 mg to about 5 g of the first therapeutic agent and about 0.1 mg to about 5 g of the second therapeutic agent and about 0.1 mg to about 5 g of the third therapeutic agent, all of which are compounded with an appropriate and convenient amount of carrier material varying from about 5 to about 95 percent of the total composition. Unit dosages will generally contain between from about 0.5 mg to about 1500 mg of the first therapeutic agent, about 0.5 mg to about 1500 mg of the second therapeutic agent, and about 0.5 mg to about 1500 mg of the third therapeutic agent. In certain embodiments, the dosage is about 25 mg, 50 mg, 100 mg, 200 mg, 300 mg, 400 mg, 500 mg, 600 mg, 800 mg, or 1000 mg, etc., up to about 1500 mg of the first therapeutic agent. In certain embodiments, the dosage is about 25 mg, 50 mg, 100 mg, 200 mg, 300 mg, 400 mg, 500 mg, 600 mg, 800 mg, or 1000 mg, etc., up to about 1500 mg of the second therapeutic agent. In certain embodiments, the dosage is about 25 mg, 50 mg, 100 mg, 200 mg, 300 mg, 400 mg, 500 mg, 600 mg, 800 mg, or 1000 mg, etc., up to about 1500 mg of the third therapeutic agent.

Dosage amount and interval may be adjusted on an individual or group basis to provide plasma levels of a particular active moiety or moieties sufficient to maintain the modulating effects or minimal effective concentration (MEC) of each of them. The MEC will vary for each compound and individual, but it can be estimated from in vitro data. Dosages necessary to achieve the MEC will depend on individual characteristics and route of administration. However, HPLC assays or bioassays can be used to determine plasma concentrations. In certain embodiments, the dosage is adjusted so that an individual loses weight at a rate of about 10% of initial weight about every 6 months. However, the rate of weigh loss for each individual may be adjusted by the treating physician based on the individual's particular needs. In certain embodiments, the dose may be decreased. In certain embodiments, the dose may be increased. Moreover, a long-term treatment regimen may include alternating period of increasing and decreasing dosage with respect to a particular compound or compounds.

### Synergism and Augmentation

The term “synergistic” refers to a combination which is more effective than the additive effects of any two or more single agents. A synergistic effect permits the effective treatment of a disease using lower amounts (doses) of individual therapy. The lower doses result in lower toxicity without reduced efficacy. In addition, a synergistic effect can result in improved efficacy. Finally, synergy may result in an improved avoidance or reduction of disease as compared to any single therapy.

Combination therapy can allow for the product of lower doses of the first therapeutic or the second therapeutic agent (referred to as “apparent one-way synergy” herein), or lower doses of both therapeutic agents (referred to as “two-way synergy” herein) than would normally be required when either drug is used alone.

Combination therapy can allow for the product of lower doses of any one of the therapeutic agents (referred to as “apparent one-way synergy” herein), or lower doses of all therapeutic agents than would normally be required when any drug is used alone.

In certain embodiments, the synergism exhibited between one or more therapeutic agent(s) and the remaining therapeutic agent(s) is such that the dosage of one of the therapeutic agents would be sub-therapeutic if administered without the dosage of the other therapeutic agents.

The terms “augmentation” or “augment” refer to combinations where one of the compounds increases or enhances therapeutic effects of another compound or compounds administered to a patient. In some instances, augmentation can result in improving the efficacy, tolerability, or safety, or any combination thereof, of a particular therapy.

In certain embodiments, the present invention relates to a pharmaceutical composition comprising a therapeutically effective dose of one or more therapeutic agent(s) together with a dose of another therapeutic agent effective to augment the therapeutic effect of the one or more therapeutic agent(s). In other embodiments, the present invention relates to methods of augmenting the therapeutic effect in a patient of one or more therapeutic agent(s) by administering another therapeutic agent to the patient.

In certain embodiments, the invention is directed in part to synergistic combinations of one or more therapeutic agent(s) in an amount sufficient to render a therapeutic effect together with the remaining therapeutic agent(s). For example, in certain embodiments a therapeutic effect is attained which is at least about 2 (or at least about 4, 6, 8, or 10) times greater than that obtained with the dose of the one or more therapeutic agent(s) alone. In certain embodiments, the synergistic combination provides a therapeutic effect which is up to about 20, 30 or 40 times greater than that obtained with the dose of the one or more therapeutic agent(s) alone. In such embodiments, the synergistic combinations display what is referred to herein as an “apparent one-way synergy”, meaning that the dose of the remaining therapeutic agent(s) synergistically potentiates the effect of the one or more therapeutic agent(s), but the dose of the one or more therapeutic agent(s) does not appear to significantly potentiate the effect of the remaining therapeutic agent(s).

In certain embodiments, the combination of active agents exhibits two-way synergism, meaning that the second therapeutic agent potentiates the effect of the first therapeutic agent, and the first therapeutic agent potentiates the effect of the second therapeutic agent. Thus, other embodiments of the invention relate to combinations of a second therapeutic agent and a first therapeutic agent where the dose of each drug is reduced due to the synergism between the drugs, and the therapeutic effect derived from the combination of drugs in reduced doses is enhanced. The two-way synergism is not always readily apparent in actual dosages due to the potency ratio of the first therapeutic agent to the second therapeutic agent. For instance, two-way synergism can be difficult to detect when one therapeutic agent displays much greater therapeutic potency relative to the other therapeutic agent.

The synergistic effects of combination therapy may be evaluated by biological activity assays. For example, the therapeutic agents are mixed at molar ratios designed to give approximately equipotent therapeutic effects based on the EC₉₀ values. Then, three different molar ratios are used for each combination to allow for variability in the estimates of relative potency. These molar ratios are maintained throughout the dilution series. The corresponding monotherapies are also evaluated in parallel to the combination treatments using the standard primary assay format. A comparison of the therapeutic effect of the combination treatment to the therapeutic effect of the monotherapy gives a measure of the synergistic effect. Further details on the design of combination analyses can be found in B E Korba (1996) Antiviral Res. 29:49. Analysis of synergism, additivity, or antagonism can be determined by analysis of the aforementioned data using the CalcuSyn™ program (Biosoft, Inc.). This program evaluates drug interactions by use of the widely accepted method of Chou and Talalay combined with a statistically evaluation using the Monte Carlo statistical package. The data are displayed in several different formats including median-effect and dose-effects plots, isobolograms, and combination index [CI] plots with standard deviations. For the latter analysis, a CI greater than 1.0 indicates antagonism and a CI less than 1.0 indicates synergism.

Compositions of the invention present the opportunity for obtaining relief from moderate to severe cases of disease. Due to the synergistic or additive or augmented effects provided by the inventive combination of the first and second therapeutic agent, it may be possible to use reduced dosages of each of therapeutic agent. Due to the synergistic or additive or augmented effects provided by the inventive combination of the first, second, and third therapeutic agents, it may be possible to use reduced dosages of each of therapeutic agent. By using lesser amounts of drugs, the side effects associated with each may be reduced in number and degree. Moreover, the inventive combinations avoid side effects to which some patients are particularly sensitive.

### PHARMACEUTICAL COMPOSITIONS AND FORMULATIONS

### Pharmaceutical Compositions

The present invention provides pharmaceutically acceptable compositions which comprise a therapeutically-effective amount of two or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a therapeutic agent in a composition of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or stearic acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the compounds found in the present compositions may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds comprised in compositions of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the compounds that the present compositions comprise include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isothionic, and the like.

In other cases, the compounds comprised in compositions of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredients which can be combined with a carrier material to produce a single dosage form will vary depending upon the host being treated, the particular mode of administration. The amount of active ingredients which can be combined with a carrier material to produce a single dosage form will generally be those amounts of the compounds which produce a therapeutic effect. Generally, out of one hundred per cent, this amount will range from about 0.1 per cent to about ninety-nine percent of active ingredients, from about 5 per cent to about 70 per cent, or from about 10 per cent to about 30 per cent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides. In certain embodiments, an aforementioned formulation renders orally bioavailable a composition of the present invention.

Methods of preparing these formulations or compositions include the step of bringing into association two or more active compounds with the carrier and, optionally, one or more accessory ingredients. In general, the formulations are prepared by uniformly and intimately bringing into association one or more active compounds with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active ingredients. A composition of the present invention may also be administered as a bolus, electuary or paste.

In solid dosage forms of the invention for oral administration (capsules, tablets, pills, dragees, powders, granules, trouches and the like), the active ingredients are mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredients therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredients only in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compositions of the invention include pharmaceutically acceptable emulsions, microemulsions, solutions, suspensions, syrups and elixirs. In addition to the active ingredients, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing the active ingredients of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a composition of this invention include powders, sprays, ointments, pastes, creams, lotions, gels, solutions, patches and inhalants. The active compounds may be mixed under sterile conditions with a pharmaceutically-acceptable carrier, and with any preservatives, buffers, or propellants which may be required.

The ointments, pastes, creams and gels may contain, in addition to the active compounds, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the active compounds, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the active compounds to the body. Such dosage forms can be made by dissolving or dispersing the active compounds in the proper medium. Absorption enhancers can also be used to increase the flux of the compounds across the skin. The rate of such flux can be controlled by either providing a rate controlling membrane or dispersing the compounds in a polymer matrix or gel.

Ophthalmic formulations, eye ointments, powders, solutions and the like, are also contemplated as being within the scope of this invention.

Pharmaceutical compositions of this invention suitable for parenteral administration comprise two or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the product of coating materials, such as lecithin, by the maintenance of the required particle size in the case of dispersions, and by The product of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The compositions comprising the two or more therapeutic agents can be, alone or in combination with other therapeutic agents, employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For parenteral application, particularly suitable are oily or aqueous solutions, as well as suspensions, emulsions, or implants, including suppositories. Ampoules are convenient unit dosages. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay release of the active ingredients. Formulations for oral use may also be presented as hard gelatin capsules wherein the active ingredients are mixed with an inert diluent.

Aqueous suspensions contain the above-identified combinations of drugs and that mixture has one or more excipients suitable as suspending agents, for example pharmaceutically acceptable synthetic gums such as hydroxypropylmethylcellulose or natural gums. Oily suspensions may be formulated by suspending the above-identified combination of drugs in a vegetable oil or mineral oil. The oily suspensions may contain a thickening agent such as beeswax or cetyl alcohol. A syrup, elixir, or the like can be used wherein a sweetened vehicle is employed. Injectable suspensions may also be prepared, in which case appropriate liquid carriers, suspending agents and the like may be employed. It is also possible to freeze-dry the active compounds and use the obtained lyophilized compounds, for example, for the preparation of products for injection.

One aspect of combination therapy pertains to a method for providing effective therapeutic treatment in humans, comprising administering an effective or sub-therapeutic amount of one or more therapeutic agent(s); and administering the remaining therapeutic agent(s) in an amount effective to augment the therapeutic effect provided by said one or more therapeutic agent(s). The therapeutic agents can be administered simultaneously or at different times, as long as the dosing intervals (or the therapeutic effects) of the therapeutic agents overlaps. In other words, according to the method of the present invention, in certain embodiments the therapeutic agents need not be administered in the same dosage form or even by the same route of administration as each other. Rather, the method is directed to the surprising synergistic and/or additive benefits obtained in humans, when therapeutically effective levels of one or more therapeutic agent(s) have been administered to a human, and, prior to or during the dosage interval for the therapeutic agent(s) or while the human is experiencing the therapeutic effect, an effective amount of other therapeutic agent(s) to augment the therapeutic effect of the original one or more therapeutic agent(s) is administered.

Another aspect of combination therapy relates to an oral solid dosage form comprising a therapeutically effective amount of one or more therapeutic agent(s) together with an amount of the remaining therapeutic agent(s) or pharmaceutically acceptable salt thereof which augments the effect of the one or more therapeutic agent(s).

In some cases, in order to prolong the effect of a drug, it is desirable to slow the absorption of the drug from subcutaneous or intramuscular injection. This may be accomplished by the product of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drugs to polymer, and the nature of the particular polymer employed, the rate of drug release can be controlled. Examples of other biodegradable polymers include poly(orthoesters) and poly(anhydrides). Depot injectable formulations are also prepared by entrapping the drugs in liposomes or microemulsions which are compatible with body tissue.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Actual dosage levels of the active ingredients in the pharmaceutical compositions of this invention may be varied so as to obtain an amount of an active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the active compounds employed in the pharmaceutical composition at levels lower than that required in order to achieve the desired therapeutic effect and gradually increase the dosage until the desired effect is achieved.

While it is possible for an active compound of the present invention to be administered alone, in certain embodiments the compound is administered as a pharmaceutical formulation (composition).

In another aspect, the present invention provides pharmaceutically acceptable compositions which comprise a therapeutically-effective amount of the active compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compounds of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutic effects of the first administered one is not entirely disappeared when the subsequent is administered.

Micelles

Recently, the pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

In one aspect of invention, the formulations contain micelles formed from a compound of the present invention and at least one amphiphilic carrier, in which the micelles have an average diameter of less than about 100 nm. Certain embodiments provide micelles having an average diameter less than about 50 nm, and certain embodiments provide micelles having an average diameter less than about 30 nm, or even less than about 20 nm.

While all suitable amphiphilic carriers are contemplated, in certain embodiments, the carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

In certain embodiments, amphiphilic carriers are saturated and monounsaturated polyethyleneglycolyzed fatty acid glycerides, such as those obtained from fully or partially hydrogenated various vegetable oils. Such oils may advantageously consist of tri-, di- and mono-fatty acid glycerides and di- and mono-polyethyleneglycol esters of the corresponding fatty acids. In certain embodiments, the fatty acid composition includes capric acid 4-10, capric acid 3-9, lauric acid 40-50, myristic acid 14-24, palmitic acid 4-14, or stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc. (produced and distributed by a number of companies in USA and worldwide).

Polymers

Hydrophilic polymers suitable for use in the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In certain embodiments, the polymers are those having a molecular weight of from about 100 or 120 daltons up to about 5,000 or 10,000 daltons, or from about 300 daltons to about 5,000 daltons. In certain embodiments, the polymer is polyethyleneglycol having a molecular weight of from about 100 to about 5,000 daltons, or having a molecular weight of from about 300 to about 5,000 daltons. In certain embodiments, the polymer is polyethyleneglycol of 750 daltons (PEG(750)). Polymers may also be defined by the number of monomers therein; in certain embodiments of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins

Cyclodextrins are cyclic oligosaccharides, consisting of 6, 7 or 8 glucose units, designated by the Greek letter α, β, or γ, respectively. Cyclodextrins with fewer than six glucose units are not known to exist. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17.beta.-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259; incorporated by reference) and Gramera, et al. (U.S. Pat. No. 3,459,731; incorporated by reference) described electroneutral cyclodextrins. Other derivatives include cyclodextrins with cationic properties [Parmeter (II), U.S. Pat. No. 3,453,257; incorporated by reference], insoluble crosslinked cyclodextrins (Solms, U.S. Pat. No. 3,420,788; incorporated by reference), and cyclodextrins with anionic properties [Parmeter (III), U.S. Pat. No. 3,426,011; incorporated by reference]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127; incorporated by reference).

Liposomes

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present invention relates to formulations comprising liposomes containing one or more of the therapeutic agents of the present invention, where the liposome membrane is formulated to provide a liposome with increased carrying capacity. Alternatively or in addition, the one or more therapeutic agents may be contained within, or adsorbed onto, the liposome bilayer of the liposome. One or more therapeutic agents may be aggregated with a lipid surfactant and carried within the liposome's internal space; in these cases, the liposome membrane is formulated to resist the disruptive effects of the active agent-surfactant aggregate.

According to one embodiment of the present invention, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present invention are in solubilized form. Aggregates of surfactant and active agent (such as emulsions or micelles containing the active agent of interest) may be entrapped within the interior space of liposomes according to the present invention. A surfactant acts to disperse and solubilize the active agents, and may be selected from any suitable aliphatic, cycloaliphatic or aromatic surfactant, including but not limited to biocompatible lysophosphatidylcholines (LPCs) of varying chain lengths (for example, from about C₁₄ to about C₂₀). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. In certain embodiments, the surfactants have CMCs in the micromolar range; higher CMC surfactants may be utilized to prepare micelles entrapped within liposomes of the present invention, however, micelle surfactant monomers could affect liposome bilayer stability and would be a factor in designing a liposome of a desired stability.

Liposomes according to the present invention may be prepared by any of a variety of techniques that are known in the art. See, e.g., U.S. Pat. No. 4,235,871; incorporated by reference; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993.

For example, liposomes of the present invention may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, one or more active agents are first dispersed by sonication in a lysophosphatidylcholine or other low CMC surfactant (including polymer grafted lipids) that readily solubilizes hydrophobic molecules. The resulting micellar suspension of one or more active agents is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See, e.g., U.S. Pat. No. 4,737,323 (Apr. 12, 1988; incorporated by reference).

Release Modifiers

The release characteristics of a formulation of the present invention depend on the encapsulating material, the concentration of encapsulated drugs, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore-forming agents which add microstructure to the matrices (i.e., water soluble compounds, such as inorganic salts and sugars) are added as particulates. The range should be between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

### Immediate/Sustained Release Combination Therapy Dosage Forms

The combination therapy may be formulated in an immediate release dosage form or a sustained release dosage form. In certain embodiments, the present invention relates to immediate release dosage forms of two or more therapeutic agents. An immediate release dosage form may be formulated as a tablet or multiparticulate which may be encapsulated. Other immediate release dosage forms known in the art can be employed. In certain embodiments, the combination of therapeutic agents may be formulated to provide for an increased duration (sustained release) of therapeutic action. These formulations, at comparable daily dosages of conventional immediate release drug, are often associated with a lower incidence or severity of adverse drug reactions; and they can also be administered at a lower daily dose than conventional oral medication while maintaining therapeutic activity.

In certain embodiments, the combination therapy can be formulated to deliver the therapeutic agents on the same or different time schedules. In certain embodiments, the therapeutic agents are administered via an oral solid dosage form that includes a sustained release carrier causing the sustained release of any one or more of the therapeutic agent(s) when the dosage form contacts gastrointestinal fluid. The sustained release dosage form may comprise a plurality of substrates which include the drugs. The substrates may comprise matrix spheroids or may comprise inert pharmaceutically acceptable beads which are coated with the drugs. The coated beads may then be overcoated with a sustained release coating comprising the sustained release carrier. The matrix spheroid may include the sustained release carrier in the matrix itself; or the matrix may comprise a normal release matrix containing the drugs, the matrix having a coating applied thereon which comprises the sustained release carrier. In other embodiments, the oral solid dosage form comprises a tablet core containing the drugs within a normal release matrix, with the tablet core being coated with a sustained release coating comprising the sustained release carrier. In further embodiments, the tablet contains the drugs within a sustained release matrix comprising the sustained release carrier. In additional embodiments, the tablet contains one or more therapeutic agent(s) within a sustained release matrix and remaining therapeutic agent(s) coated into the tablet as an immediate release layer.

The term “sustained release” is defined for purposes of the present invention as the release of the therapeutic agent from the formulation at such a rate that blood (e.g., plasma) concentrations (levels) are maintained within the therapeutic range (above the minimum effective analgesic concentration or “MEAC”) but below toxic levels over a period of time of about 12 hours or longer.

The therapeutic agents can be formulated as a controlled or sustained release oral formulation in any suitable tablet, coated tablet or multiparticulate formulation known to those skilled in the art. The sustained release dosage form may optionally include a sustained released carrier which is incorporated into a matrix along with the active agents, or which is applied as a sustained release coating.

The sustained release dosage form may include one or more therapeutic agent in sustained release form and the remaining therapeutic agent(s) in the sustained release form or in immediate release form. One or more therapeutic agent may be incorporated into the sustained release matrix along with another therapeutic agent; one or more therapeutic agent may be incorporated into the sustained release coating; incorporated as a separated sustained release layer or immediate release layer; or may be incorporated as a powder, granulation, etc., in a gelatin capsule with the substrates of the present invention. Alternatively, the sustained release dosage form may have one or more therapeutic agent in the sustained release form and the remaining therapeutic agent(s) in the sustained release form or immediate release form.

An oral dosage form according to the invention may be provided as, for example, granules, spheroids, beads, pellets (hereinafter collectively referred to as “multiparticulates”) and/or particles. An amount of the multiparticulates which is effective to provide the desired dose of the therapeutic agents over time may be placed in a capsule or may be incorporated in any other suitable oral solid form. In one certain embodiments of the present invention, the sustained release dosage form comprises such particles containing or comprising one or more active ingredients, wherein the particles have diameter from about 0.1 mm to about 2.5 mm, or from about 0.5 mm to about 2 mm.

In certain embodiments, the particles comprise normal release matrixes containing one or more therapeutic agent with the remaining therapeutic agent(s). These particles are then coated with the sustained release carrier in embodiments where one or more therapeutic agent is immediately released, one or more therapeutic agent may be included in separate normal release matrix particles, or may be co-administered in a different immediate release composition which is either enveloped within a gelatin capsule or is administered separately. In other embodiments, the particles comprise inert beads which are coated with the remaining therapeutic agent(s) with one or more therapeutic agent. Thereafter, a coating comprising the sustained release carrier is applied onto the beads as an overcoat.

The particles may be film coated with a material that permits release of the active agents at a sustained rate in an aqueous medium. The film coat is chosen so as to achieve, in combination with the other stated properties, a desired in vitro release rate. The sustained release coating formulations of the present invention should be capable of producing a strong, continuous film that is smooth and elegant, capable of supporting pigments and other coating additives, non-toxic, inert, and tack-free.

Coatings

The dosage forms of the present invention may optionally be coated with one or more materials suitable for the regulation of release or for the protection of the formulation. In one embodiment, coatings are provided to permit either pH-dependent or pH-independent release, e.g., when exposed to gastrointestinal fluid. A pH-dependent coating serves to release any of the active agent(s) in the desired areas of the gastro-intestinal (GI) tract, e.g., the stomach or small intestine, such that an absorption profile is provided which is capable of providing at least about twelve hours or up to twenty-four hours of therapeutic benefit to a patient. When a pH-independent coating is desired, the coating is designed to achieve optimal release regardless of pH-changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine. In certain embodiments, one or more therapeutic agent(s) is released in one area of the GI tract and the remaining therapeutic agent(s) is released in a second area of the GI tract. In certain embodiments, the therapeutic agents are released in nearly equal amounts at the same location in the GI tract.

Formulations according to the invention that utilize pH-dependent coatings to obtain formulations may also impart a repeat-action effect whereby unprotected drug is coated over an enteric coat and is released in the stomach, while the remainder, being protected by the enteric coating, is released further down the gastrointestinal tract. Coatings which are pH-dependent may be used in accordance with the present invention include shellac, cellulose acetate phthalate (CAP), polyvinyl acetate phthalate (PVAP), hydroxypropylmethylcellulose phthalate, and methacrylic acid ester copolymers, zein, and the like. Thus, one aspect of the present invention relates to a formulation wherein one or more therapeutic agent(s) is coated over the enteric coat and released into the stomach while the remaining therapeutic agent(s) is protected by the enteric coating and is released further down the GI tract.

In certain embodiments, the substrate (e.g., tablet core bead, matrix particle) containing one or more therapeutic agent(s) (with or without the remaining therapeutic agent(s)) is coated with a hydrophobic material selected from (i) an alkylcellulose; (ii) an acrylic polymer; or (iii) mixtures thereof. The coating may be applied in the form of an organic or aqueous solution or dispersion. The coating may be applied to obtain a weight gain from about 2 to about 25% of the substrate in order to obtain a desired sustained release profile. Such formulations are described, e.g., in detail in U.S. Pat. Nos. 5,273,760 and 5,286,493; both incorporated by reference. Other examples of sustained release formulations and coatings which may be used in accordance with the present invention include U.S. Pat. Nos. 5,324,351; 5,356,467, and 5,472,712; all incorporated by reference.

Alkylcellulose Polymers

Cellulosic materials and polymers, including alkylcelluloses, provide hydrophobic materials well suited for coating the formulations according to the invention. Simply by way of example, one alkylcellulosic polymer is ethylcellulose, although the artisan will appreciate that other cellulose or alkylcellulose polymers may be readily employed, singly or in any combination, as all or part of a hydrophobic coating.

One commercially-available aqueous dispersion of ethylcellulose is Aquacoat® (FMC Corp., Philadelphia, Pa., U.S.A.). Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat® with a suitable plasticizer prior to use.

Another aqueous dispersion of ethylcellulose is commercially available as Surelease® (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

Acrylic Polymers

In other embodiments of the present invention, the hydrophobic material comprising the controlled release coating is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In certain embodiments, the acrylic polymer is comprised of one or more ammonio methacrylate copolymers. Ammonio methacrylate copolymers are well known in the art, and are copolymers of acrylic and methacrylic acid esters with a low content of quaternary ammonium groups. In order to obtain a desirable dissolution profile, it may be necessary to incorporate in a coating two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit® from Rohm Tech, Inc. There are several different types of Eudragit®. For example, Eudragit® E is an example of a methacrylic acid copolymer which swells and dissolves in acidic media. Eudragit® L is a methacrylic acid copolymer which does not swell at about pH<5.7 and is soluble at about pH>6. Eudragit® S does not swell at about pH<6.5 and is soluble at about pH>7. Eudragit® RL and Eudragit® RS are water swellable, and the amount of water absorbed by these polymers is pH-dependent, however, dosage forms coated with Eudragit® RL and RS are pH-independent.

In certain embodiments, the acrylic coating comprises a mixture of two acrylic resin lacquers commercially available from Rohm Pharma under the Tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

The Eudragit® RL/RS dispersions of the present invention may be mixed together in any desired ratio in order to ultimately obtain a sustained release formulation having a desirable dissolution profile. Desirable sustained release formulations may be obtained, for instance, from a retardant coating derived from 100% Eudragit® RL, 50% Eudragit® RL and 50% Eudragit® RS, and 10% Eudragit® RL:Eudragit® 90% RS. Of course, one skilled in the art will recognize that other acrylic polymers may also be used, such as, for example, Eudragit® L.

Plasticizers

In embodiments of the present invention where the coating comprises an aqueous dispersion of a hydrophobic material, the inclusion of an effective amount of a plasticizer in the aqueous dispersion of hydrophobic material will further improve the physical properties of the sustained release coating. For example, because ethylcellulose has a relatively high glass transition temperature and does not form flexible films under normal coating conditions, a plasticizer may be incorporated into an ethylcellulose coating containing sustained release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate can be a plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate can be a plasticizer for the aqueous dispersions of ethyl cellulose of the present invention.

It has further been found that the addition of a small amount of talc reduces the tendency of the aqueous dispersion to stick during processing, and acts as a polishing agent.

Processes for Preparing Coated Beads

When the aqueous dispersion of hydrophobic material is used to coat inert pharmaceutical beads such as nu pariel 18/20 beads, a plurality of the resultant stabilized solid controlled release beads may thereafter be placed in a gelatin capsule in an amount sufficient to provide an effective controlled release dose when ingested and contacted by an environmental fluid, e.g., gastric fluid or dissolution media.

The stabilized controlled release bead formulations of the present invention slowly release the therapeutically active agent, e.g., when ingested and exposed to gastric fluids, and then to intestinal fluids. The controlled release profile of the formulations of the invention can be altered, for example, by varying the amount of overcoating with the aqueous dispersion of hydrophobic material, altering the manner in which the plasticizer is added to the aqueous dispersion of hydrophobic material, by varying the amount of plasticizer relative to hydrophobic material, by the inclusion of additional ingredients or excipients, by altering the method of manufacture, etc. The dissolution profile of the ultimate product may also be modified, for example, by increasing or decreasing the thickness of the retardant coating.

Spheroids or beads coated with one or more therapeutically active agent are prepared, e.g., by dissolving the one or more therapeutically active agent in water and then spraying the solution onto a substrate, for example, nu pariel 18/20 beads, using a Wuster insert. Optionally, additional ingredients are also added prior to coating the beads in order to assist the binding of the active agents to the beads, and/or to color the solution, etc. For example, a product which includes hydroxypropylmethylcellulose, etc. with or without colorant (e.g., Opadry®, commercially available from Colorcon, Inc.) may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic controlled release coating. An example of a suitable barrier agent is one which comprises hydroxypropylmethylcellulose. However, any film-former known in the art may be used. The barrier agent may or may not affect the dissolution rate of the final product.

The beads may then be overcoated with an aqueous dispersion of the hydrophobic material. The aqueous dispersion of hydrophobic material may further include an effective amount of plasticizer, e.g., triethyl citrate. Pre-formulated aqueous dispersions of ethylcellulose, such as Aquacoat® or Surelease®, may be used. If Surelease® is used, it is not necessary to separately add a plasticizer. Alternatively, pre-formulated aqueous dispersions of acrylic polymers such as Eudragit® can be used.

The coating solutions of the present invention may contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color may be added to Aquacoat® via the product of alcohol or propylene glycol based color dispersions, milled aluminum lakes and opacifiers such as titanium dioxide by adding color with shear to water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retard effect of the coating.

The plasticized aqueous dispersion of hydrophobic material may be applied onto the substrate comprising the one or more therapeutically active agent by spraying using any suitable spray equipment known in the art. In certain embodiments, a Wurster fluidized-bed system is used in which an air jet, injected from underneath, fluidizes the core material and effects drying while the acrylic polymer coating is sprayed on. A sufficient amount of the aqueous dispersion of hydrophobic material to obtain a predetermined controlled release of said therapeutically active agents when said coated substrate is exposed to aqueous solutions, e.g., gastric fluid, is applied, taking into account the physical characteristics of the therapeutically active agents, the manner of incorporation of the plasticizer, etc. After coating with the hydrophobic material, a further overcoat of a film-former, such as Opadry®, is optionally applied to the beads. This overcoat is provided, if at all, in order to substantially reduce agglomeration of the beads.

The release of the therapeutically active agent from the controlled release formulation of the present invention can be further influenced, i.e., adjusted to a desired rate, by the addition of one or more release-modifying agents, or by providing one or more passageways through the coating. The ratio of hydrophobic material to water soluble material is determined by, among other factors, the release rate required and the solubility characteristics of the materials selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose.

The sustained release coatings of the present invention can also include erosion-promoting agents such as starch and gums.

The sustained release coatings of the present invention can also include materials useful for making microporous lamina in the environment of use, such as polycarbonates comprised of linear polyesters of carbonic acid in which carbonate groups reoccur in the polymer chain. The release-modifying agent may also comprise a semi-permeable polymer.

In certain embodiments, the release-modifying agent is selected from hydroxypropylmethylcellulose, lactose, metal stearates, and mixtures of any of the foregoing.

The sustained release coatings of the present invention may also include an exit means comprising at least one passageway, orifice, or the like. The passageway may be formed by such methods as those disclosed in U.S. Pat. Nos. 3,845,770; 3,916,889; 4,063,064; and 4,088,864; all incorporated by reference. The passageway can have any shape such as round, triangular, square, elliptical, irregular, etc.

Matrix Bead Formulations

In other embodiments of the present invention, the controlled release formulation is achieved via a matrix having a controlled release coating as set forth above. The present invention may also utilize a controlled release matrix that affords in-vitro dissolution rates of the active agents and that releases the active agents in a pH-dependent or pH-independent manner. The materials suitable for inclusion in a controlled release matrix will depend on the method used to form the matrix.

For example, a matrix, in addition to one or more of the active agents, may include: (1) Hydrophilic and/or hydrophobic materials, such as gums, cellulose ethers, acrylic resins, protein derived materials; the list is not meant to be exclusive, and any pharmaceutically acceptable hydrophobic material or hydrophilic material which is capable of imparting controlled release of the active agents and which melts (or softens to the extent necessary to be extruded) may be used in accordance with the present invention. (2) Digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes, and stearyl alcohol; and polyalkylene glycols.

The hydrophobic material may be selected from the group consisting of alkylcelluloses, acrylic and methacrylic acid polymers and copolymers, shellac, zein, hydrogenated castor oil, hydrogenated vegetable oil, or mixtures thereof. In certain embodiments of the present invention, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), and glycidyl methacrylate copolymers. In other embodiments, the hydrophobic material is selected from materials such as hydroxyalkylcelluloses such as hydroxypropylmethylcellulose and mixtures of the foregoing.

Hydrophobic materials are water-insoluble with more or less pronounced hydrophilic and/or hydrophobic trends. Generally, the hydrophobic materials useful in the invention have a melting point from about 30° C. to about 200° C., or from about 45° C. to about 90° C. Specifically, the hydrophobic material may comprise natural or synthetic waxes, fatty alcohols (such as lauryl, myristyl, stearyl, cetyl or cetostearyl alcohol), fatty acids, including but not limited to fatty acid esters, fatty acid glycerides (mono-, di-, and tri-glycerides), hydrogenated fats, hydrocarbons, normal waxes, stearic aid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material which is normally solid at room temperature and has a melting point of from about 30° C. to about 100° C.

Suitable hydrophobic materials which may be used in accordance with the present invention include digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and natural and synthetic waxes. Hydrocarbons may have a melting point of between about 25° C. and about 90° C. Of the long chain hydrocarbon materials, fatty (aliphatic) alcohols may be used in certain embodiments. The oral dosage form may contain up to 60% (by weight) of at least one digestible, long chain hydrocarbon.

In certain instances, a combination of two or more hydrophobic materials is included in the matrix formulations. If an additional hydrophobic material is included, it may be selected from natural and synthetic waxes, fatty acids, fatty alcohols, and mixtures of the same. Examples include beeswax, carnauba wax, stearic acid and stearyl alcohol. This list is not meant to be exclusive.

One particular suitable matrix comprises at least one water soluble hydroxyalkyl cellulose, at least one C₁₂-C₃₆, or C₁₄-C₂₂, aliphatic alcohol and, optionally, at least one polyalkylene glycol. The at least one hydroxyalkyl cellulose may be a hydroxy (C₁ to C₆) alkyl cellulose, such as hydroxypropylcellulose, hydroxypropylmethylcellulose and, especially, hydroxyethylcellulose. The amount of the at least one hydroxyalkyl cellulose in the present oral dosage form will be determined, inter alia, by the precise rate of release desired for the therapeutic agent. The at least one aliphatic alcohol may be, for example, lauryl alcohol, myristyl alcohol or stearyl alcohol. In certain embodiments of the present oral dosage form, however, the at least one aliphatic alcohol is cetyl alcohol or cetostearyl alcohol. The amount of the at least one aliphatic alcohol in the present oral dosage form will be determined, as above, by the precise rate of release desired for the therapeutic agents. It will also depend on whether at least one polyalkylene glycol is present in or absent from the oral dosage form. In the absence of at least one polyalkylene glycol, the oral dosage form may contain between 20% and 50% (by wt) of the at least one aliphatic alcohol. When at least one polyalkylene glycol is present in the oral dosage form, then the combined weight of the at least one aliphatic alcohol and the at least one polyalkylene glycol may constitute between 20% and 50% (by wt) of the total dosage.

In one embodiment, the ratio of, e.g., the at least one hydroxyalkyl cellulose or acrylic resin to the at least one aliphatic alcohol/polyalkylene glycol determines, to a considerable extent, the release rate of the active agent from the formulation. The ratio of the at least one hydroxyalkyl cellulose to the at least one aliphatic alcohol/polyalkylene glycol may be between 1:2 and 1:4, or between 1:3 and 1:4.

The at least one polyalkylene glycol may be, for example, polypropylene glycol or polyethylene glycol. The number average molecular weight of the at least one polyalkylene glycol may be between about 1,000 and about 15,000, or between about 1,500 and about 12,000. Another suitable controlled release matrix would comprise an alkylcellulose (especially ethyl cellulose), a C₁₂ to C₃₆ aliphatic alcohol and, optionally, a polyalkylene glycol. In certain embodiments, the matrix includes a pharmaceutically acceptable combination of at least two hydrophobic materials. In addition to the above ingredients, a controlled release matrix may also contain suitable quantities of other materials, e.g., diluents, lubricants, binders, granulating aids, colorants, flavorants and glidants that are conventional in the pharmaceutical art.

### Processes for Preparing Controlled-Release Dosage Forms

In order to facilitate the preparation of a solid, controlled release, oral dosage form according to this invention, any method of preparing a matrix formulation known to those skilled in the art may be used. For example incorporation in the matrix may be effected, for example, by (a) forming granules comprising at least one water soluble hydroxyalkyl cellulose and the one or more active agents; (b) mixing the hydroxyalkyl cellulose containing granules with at least one C₁₂-C₃₆ aliphatic alcohol; and (c) optionally, compressing and shaping the granules. The granules may be formed by wet granulating the hydroxyalkyl cellulose/active agent with water. In one embodiment of this process, the amount of water added during the wet granulation step may be between about 1.5 and about 5 times, or between about 1.75 and about 3.5 times, the dry weight of the active agent.

In yet other alternative embodiments, a spheronizing agent, together with one or more active ingredients can be spheronized to form spheroids. Microcrystalline cellulose may be used. A suitable microcrystalline cellulose is, for example, the material sold as Avicel PH 101 (Trade Mark, FMC Corporation). In such embodiments, in addition to the one or more active ingredients and spheronizing agent, the spheroids may also contain a binder. Suitable binders, such as low viscosity, water soluble polymers, will be well known to those skilled in the pharmaceutical art. Water soluble hydroxy lower alkyl cellulose, such as hydroxypropylcellulose, may be used. Additionally (or alternatively) the spheroids may contain a water insoluble polymer, especially an acrylic polymer, an acrylic copolymer, such as a methacrylic acid-ethyl acrylate copolymer, or ethyl cellulose. In such embodiments, the sustained release coating will generally include a hydrophobic material such as (a) a wax, either alone or in admixture with a fatty alcohol; or (b) shellac or zein.

Melt Extrusion Matrix

Sustained release matrices can also be prepared via melt-granulation or melt-extrusion techniques. Generally, melt-granulation techniques involve melting a normally solid hydrophobic material, e.g., a wax, and incorporating a powdered drug therein. To obtain a sustained release dosage form, it may be necessary to incorporate an additional hydrophobic substance, e.g., ethylcellulose or a water-insoluble acrylic polymer, into the molten wax hydrophobic material. Examples of sustained release formulations prepared via melt-granulation techniques are found in U.S. Pat. No. 4,861,598; incorporated by reference.

The additional hydrophobic material may comprise one or more water-insoluble wax-like thermoplastic substances possibly mixed with one or more wax-like thermoplastic substances being less hydrophobic than said one or more water-insoluble wax-like substances. In order to achieve constant release, the individual wax-like substances in the formulation should be substantially non-degradable and insoluble in gastrointestinal fluids during the initial release phases. Useful water-insoluble wax-like substances may be those with a water-solubility that is lower than about 1:5,000 (w/w).

In addition to the above ingredients, a sustained release matrix may also contain suitable quantities of other materials, e.g., diluents, lubricants, binders, granulating aids, colorants, flavorants and glidants that are conventional in the pharmaceutical art. The quantities of these additional materials will be sufficient to provide the desired effect to the desired formulation. In addition to the above ingredients, a sustained release matrix incorporating melt-extruded multiparticulates may also contain suitable quantities of other materials, e.g., diluents, lubricants, binders, granulating aids, colorants, flavorants and glidants that are conventional in the pharmaceutical art in amounts up to about 50% by weight of the particulate if desired.

Specific examples of pharmaceutically acceptable carriers and excipients that may be used to formulate oral dosage forms are described in the Handbook of Pharmaceutical Excipients, American Pharmaceutical Association (1986).

Melt Extrusion Multiparticulates

The preparation of a suitable melt-extruded matrix according to the present invention may, for example, include the steps of blending the active agents, together with at least one hydrophobic material and optionally the additional hydrophobic material to obtain a homogeneous mixture. The homogeneous mixture is then heated to a temperature sufficient to at least soften the mixture sufficiently to extrude the same. The resulting homogeneous mixture is then extruded to form strands. The extrudate may be cooled and cut into multiparticulates by any means known in the art. The strands are cooled and cut into multiparticulates. The multiparticulates are then divided into unit doses. The extrudate may have a diameter of from about 0.1 to about 5 mm and provides sustained release of the therapeutically active agent for a time period of from about 8 to about 24 hours.

An optional process for preparing the melt extrusions of the present invention includes directly metering into an extruder a hydrophobic material, the therapeutically active agents, and an optional binder; heating the homogenous mixture; extruding the homogenous mixture to thereby form strands; cooling the strands containing the homogeneous mixture; cutting the strands into particles having a size from about 0.1 mm to about 12 mm; and dividing said particles into unit doses. In this aspect of the invention, a relatively continuous manufacturing procedure is realized.

The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

The melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, optionally within a range of similar size and/or shape and containing one or more active agents and one or more excipients, optionally including a hydrophobic material as described herein. In this regard, the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agents without the need of a spheronization step.

In one embodiment, oral dosage forms are prepared to include an effective amount of melt-extruded multiparticulates within a capsule. For example, a plurality of the melt-extruded multiparticulates may be placed in a gelatin capsule in an amount sufficient to provide an effective sustained release dose when ingested and contacted by gastric fluid.

In another embodiment, a suitable amount of the multiparticulate extrudate is compressed into an oral tablet using conventional tableting equipment using standard techniques. Techniques and compositions for making tablets (compressed and molded), capsules (hard and soft gelatin) and pills are also described in Remington's Pharmaceutical Sciences, (Arthur Osol, editor), 1553-1593 (1980).

In yet another embodiment, the extrudate can be shaped into tablets as set forth in U.S. Pat. No. 4,957,681 (Klimesch, et. al.); incorporated by reference.

Optionally, the sustained release melt-extruded multiparticulate systems or tablets can be coated, or the gelatin capsule can be further coated, with a sustained release coating such as the sustained release coatings described above. Such coatings may include a sufficient amount of hydrophobic material to obtain a weight gain level from about 2 to about 30 percent, although the overcoat may be greater depending upon the physical properties of the particular active agent utilized and the desired release rate, among other things.

The melt-extruded unit dosage forms of the present invention may further include combinations of melt-extruded multiparticulates containing one or more of the therapeutically active agents disclosed above before being encapsulated. Furthermore, the unit dosage forms can also include an amount of one or more immediate release therapeutically active agents for prompt therapeutic effect. The immediate release therapeutically active agent(s) may be incorporated, e.g., as separate pellets within a gelatin capsule, or may be coated on the surface of the multiparticulates after preparation of the dosage forms (e.g., controlled release coating or matrix-based). The unit dosage forms of the present invention may also contain a combination of controlled release beads and matrix multiparticulates to achieve a desired effect.

The sustained release formulations of the present invention may slowly release the therapeutically active agents, e.g., when ingested and exposed to gastric fluids, and then to intestinal fluids. The sustained release profile of the melt-extruded formulations of the invention can be altered, for example, by varying the amount of retardant, i.e., hydrophobic material, by varying the amount of plasticizer relative to hydrophobic material, by the inclusion of additional ingredients or excipients, by altering the method of manufacture, etc.

In other embodiments of the invention, the melt extruded material is prepared without the inclusion of the therapeutically active agents, which are added thereafter to the extrudate. Such formulations typically will have the therapeutically active agents blended together with the extruded matrix material, and then the mixture would be tableted in order to provide a slow release formulation. Such formulations may be advantageous, for example, when the therapeutically active agents included in the formulation are sensitive to temperatures needed for softening the hydrophobic material and/or the retardant material.

### Medical Devices

In addition to pharmaceutical solutions for the treatment of obesity, a variety of medical devices for use in the treatment of obesity has been developed, and is being introduced into clinical practice. While many of these devices are still in clinical trials, researchers remain optimistic regarding their prospects as components of low-severity, high-efficacy treatments for obesity. Moreover, the importance of these devices is magnified by the fact that many severely obese patients are not ideal candidates for surgical intervention. Therefore, such devices promise to provide new treatment options for patients suffering from obesity and other metabolic conditions, and in some cases may offer valuable alternatives to more invasive surgical approaches.

Endoluminal sleeves are one example of a device developed for the treatment of obesity. The sleeve creates a physical barrier between ingested food and the intestinal wall, thereby changing the metabolic pathway by controlling how food moves through the digestive system. This mechanical bypass of the small intestine mimics the effects on a patient's metabolism of gastric bypass surgery, often resulting in profound weight loss and remission of type 2 diabetes. The device can be implanted and removed endoscopically (via the mouth), without the need for surgical intervention.

Intragastric balloons are a second example. An intragastric balloon is designed to occupy volume within the stomach such that a smaller volume of food results in a feeling of satiety. Intragastric balloons currently on the market are not fixed in the stomach and, consequently, can lead to complications such as obstruction and mucosal erosion. To avoid these complications, the balloons are removed after a maximum of six months. One study found that the average excess weight loss was about 48.3% after one year. However, the patients reported occurrences of nausea and vomiting; and a smaller number of patients suffered from epigastric pain. Furthermore, balloon impaction occurred in about 0.6% of patients. A balloon which is fixed to the wall of the stomach could potentially improve the overall safety and efficacy of this approach, and allow longer-term implantation.

Devices have also been developed that reduce or reallocate the volume of a patient's gastrointestinal lumen. An example of such a device comprises an anchor that, once deployed, reduces a cross-sectional area within the GI track of a patient. A number of related devices in this class, such as staples, blind staples, bands, clips, tags, adhesives, and screws, have been used to reduce or reallocate the volume of a patient's stomach, specifically.

Another approach involves the use of electrical current to stimulate the stomach or certain nerves of the digestive tract. Medtronic (Minneapolis) has developed a battery-powered, stopwatch-size gastric pacemaker (similar to a cardiac pacemaker) that causes the stomach to contract, sending signals of satiety to the appetite center in the brain. The gastric pacemaker is implanted under the skin of the abdomen with electric wires placed on the wall of the stomach. Additionally, the electricity will modify eating behavior by regulating appetite signals. Moreover, the gastric pacemaker may also work to boost metabolism, which can lead to further weight loss.

An implant that uses electrical charges to inhibit the main nerve (vagus nerve) leading to the stomach has also been developed. In this case, the electrical charge may slow down digestion; for example, due to the stimulation the stomach would not register that presence of food and, therefore, would not initiate the digestive process. By down-regulating the activity of the vagus nerve, the technology simultaneously controls multiple major biological functions related to obesity, including food intake, hunger perception and digestion. Furthermore, the modulation is reversible, and the therapy can be adjusted and programmed to meet an individual patient's treatment needs.

Deep-brain-stimulation technology is also being developed as a possible treatment for obesity, which uses tiny electrodes implanted in specific areas of the brain to affect behavior, movement and other functions. Brain stimulation technology is currently approved in the United States to treat movement disorders, such as Parkinson's disease, and is being studied to treat obsessive compulsive disorder and severe depression.

Also being examined are devices that deliver an electrical charge to the same parts of the nervous system that are activated by exercise, which is known to be associated with increased metabolism. Such devices may be able to help people lose weight by boosting their metabolism.

### Medical Procedures

Normally, after food is chewed and swallowed, it moves down the esophagus to the stomach, where strong acid continues the digestive process. The stomach can hold about three pints of food at one time. Then the stomach contents moves to the duodenum, the first segment of the small intestine, where bile and pancreatic juice speed up digestion. Most of the iron and calcium in the food we eat is absorbed in the duodenum. The jejunum and ileum, the remaining two segments of the nearly 20 feet of small intestine, complete the absorption of almost all calories and nutrients. The food particles that cannot be digested in the small intestine are stored in the large intestine until eliminated.

### Weight Loss Surgery Options

Notably, severe obesity is a chronic condition that is difficult to treat effectively through diet and exercise alone. Bariatric surgery is an option for people who are severely obese and cannot lose weight by traditional means or who suffer from serious obesity-related health problems. The operation promotes weight loss and reduces the risk of type 2 diabetes by restricting food intake and, in some forms, interrupts or interferes with the digestive process described above to prevent the absorption of some calories and nutrients. Recent studies suggest that bariatric surgery may also have a favorable impact on mortality rates in severely obese patients. The best outcomes are achieved when bariatric surgery is followed with healthy eating behaviors and regular physical activity. Therefore, patients who undergo bariatric surgery should also commit to a lifetime of healthy eating and regular physical activity. These healthy habits help ensure that the weight loss from surgery is successfully maintained.

Bariatric surgery may be performed through “open” approaches, which make abdominal incisions in the traditional manner, or by laparoscopy. With a laparoscopic approach, sophisticated instruments are inserted through ½-inch incisions and guided by a small camera that sends images to a television monitor. Most bariatric surgery today is performed laparoscopically because it requires a smaller incision, creates less tissue damage, leads to earlier discharge from the hospital, and has fewer associated complications, especially postoperative hernias. However, not all patients are suitable for laparoscopy. Patients who are extremely obese, who have had previous abdominal surgery, or who have complicating medical problems may require an open surgical approach.

The American Society for Bariatric Surgery defines two basic approaches that weight-loss surgery takes to achieve change:

1. Restrictive procedures that decrease food intake; and

2. Malabsorptive procedures that alter digestion, thus causing the food to be poorly digested and incompletely absorbed so that it is eliminated in the stool after only partial digestion.

Four types of bariatric operations are commonly offered in the United States: adjustable gastric band (AGB); Roux-en-Y gastric bypass (RYGB); gastric sleeve (GS); and biliopancreatic bypass with a duodenal switch (BPD). Each procedure has its own benefits and risks. The optimal operation for a particular patient is chosen based on the inherent benefits and risks of the surgeries, along with many other factors, including BMI, eating behaviors, obesity-related health conditions, and any previous operations.

Restrictive Procedures

Adjustable Gastric Band (AGB)

Adjustable gastric band (AGB) works primarily by decreasing food intake. It is a purely restrictive surgical procedure in which a band is placed around the upper most part of the stomach. This band divides the stomach into two portions, one smaller and one larger portion. The outlet size of the band is controlled by a circular balloon inside the band that can be inflated or deflated with saline solution to meet the needs of the patient. Because the volume of the first portion of the stomach is decreased, most patients feel full more quickly. Digestion occurs through the normal digestive process. Advantages of this procedure include: restriction of the amount of food that can be consumed at a meal; food consumed passes through the digestive tract in the usual order allowing it to be fully absorbed into the body; multiple studies involving over 3,000 patients showed excess weight loss from about 28-87%, with a minimum of two year postoperative follow-up; the band can be adjusted to increase or decrease restriction; and the surgery can be reversed. However, as with any surgical procedure, there are a number of risks associated with AGB, including: perforation or tearing in the stomach wall (which may necessitate another operation); access port leakage or twisting (which may necessitate another operation); the procedure may not succeed in providing the patient a sense of fullness after consumption of smaller meals; nausea and vomiting; outlet obstruction; pouch dilatation; and band migration/slippage.

Vertical Banded Gastroplasty (VBG)

Vertical banded gastroplasty (VBG) is also a restrictive procedure. In this procedure the upper stomach near the esophagus is stapled vertically for about 2½ inches (˜6 cm) to create a smaller stomach pouch. The outlet from the pouch is restricted by a band or ring that slows the emptying of the food, thereby creating a sense of fullness after consumption of smaller meals. Advantages of VBG include: a reduced amount of well-chewed food enters and passes through the digestive tract; nutrients, vitamins, and calories are fully absorbed into the body; and studies have shown that many patients maintain 50% of targeted excess weight loss after 10 years. Of course, VBG also carries with it certain inherent risks, including: staple-line disruption (which may result in leakage or serious infection, requiring prolonged hospitalization with antibiotic treatment and/or additional operations); staple-line disruption may lead to long-term weight gain; obstruction or perforation of the band (which may require surgical intervention); failure to provide the patient with the necessary feeling of satisfaction after consumption of smaller meals; pouch stretching; band breakage or migration; and around 40% of patients lose less than half of their excess body weight.

Gastric Sleeve (GS)

The vertical gastric sleeve (GS) gastrectomy is a restrictive form of weight loss surgery in which approximately 85% of the stomach is removed, leaving a cylindrical or sleeve-shaped stomach with a volume ranging from about 60 to 150 cc, depending upon the patient and his or her goals in the procedure. Unlike many other forms of bariatric surgery, in this procedure the outlet valve and the nerves to the stomach remain intact and, while the stomach is drastically reduced in size, its function is preserved. Because the new stomach continues to function normally there are far fewer restrictions on the foods that can be consumed after surgery, although the quantity of food ingested will be considerably reduced. Additionally, the removal of the majority of the stomach results in the virtual elimination of the hunger-stimulating hormones produced in the stomach.

Perhaps the greatest advantage of the gastric sleeve is that it does not involve any bypass of the intestinal tract. Patients, therefore, do not suffer the complications of intestinal bypass, such as intestinal obstruction, anemia, osteoporosis, vitamin deficiency and protein deficiency. Moreover, the surgery is suitable for patients who are already suffering from anemia, Crohn's disease and a variety of other conditions that would place them at high risk for other procedures that did involve intestinal bypass. In general, the vertical sleeve gastrectomy is best suited to individuals who are either extremely overweight or whose overall medical condition would rule out other forms of surgery. In the former cohort of patients, a vertical sleeve gastrectomy would typically be the first stage of a two-stage surgical plan, followed by further bariatric surgical intervention once the patient's weight has fallen sufficiently to permit other forms of surgery.

Particular advantages of GS include: the stomach functions normally, aside from its reduced volume; involves removal of a major part of the stomach, resulting in decreased production of hormones responsible for stimulating hunger; the pylorus is retained, thereby avoiding contents-dumping; minimization of ulcer development; minimization of certain deleterious side effects, including anemia, intestinal obstruction or blockage, osteoporosis, and protein and vitamin deficiencies; suitability for patients with conditions that would place them at unacceptably high risk in other forms of bariatric surgery; and laparoscopic solution for patients with a particularly high body mass index (BMI). Disadvantages of the gastric sleeve gastrectomy, however, include: potential disappointing weight loss or weight regain; high BMI patients often require follow-up weight-loss surgery to achieve their goals; strict adherence to dietary guidelines following the procedure is required for optimal results; and the procedure is irreversible.

Malabsorptive Procedures

In recent years, improved clinical mastery of approaches combining restrictive and malabsorptive procedures has increased the number of effective weight-loss-surgery options for thousands of patients. A malabsorption approach to intervention means that food is delayed in mixing with bile and pancreatic juices that aid in the absorption of nutrients. The result is an earlier sense of fullness, combined with a sense of satisfaction that reduces the desire to eat.

While the operations classified as “malabsorptive” also reduce the size of the stomach, the stomach pouch created is larger than the stomach size associated with other procedures. The goals of malabsorptive procedures are to restrict the amount of food consumed and alter the normal digestive process. The anatomy of the small intestine is changed to divert the bile and pancreatic juices so they meet the ingested food around the middle or the end of the small intestine. Consequently, in the approaches summarized below, absorption of nutrients and calories is reduced. However, the procedures differ in how and when the digestive juices (i.e., bile) come into contact with the food.

Potential early complications associated with these operations include bleeding, infection, leaks from the site where the intestines are sewn together, and blood clots in the legs that may progress to the lungs and heart. Examples of complications that may occur later include malnutrition, especially in patients who do not take their prescribed vitamins and minerals. Because the duodenum is bypassed in each of these procedures, poor absorption of iron and calcium can result in the lowering of total body iron and a predisposition to iron-deficiency anemia. This side effect is a particular concern for patients who experience chronic blood loss during excessive menstrual flow or bleeding hemorrhoids. Women, already at risk for osteoporosis that can occur after menopause, should be aware of the potential for heightened bone-calcium loss.

Malnutrition, if not diagnosed and addressed promptly, may result in various diseases, such as pellagra, beri beri, and kwashiorkor, along with temporary or permanent damage to the nervous system. Other late complications include strictures (narrowing of the sites where the intestine is joined) and hernias. Patient who have had bariatric surgery are at heightened risk for two kinds of hernias. An incisional hernia is a weakness that sticks out from the abdominal wall's fascia (connective tissue) and may cause a blockage in the bowel. An internal hernia occurs when the small bowel is displaced into pockets in the lining of the abdomen; these pockets are created when the intestines are sewn together. Internal hernias are considered more dangerous than incisional hernias, the former requiring prompt intervention to avoid serious complications.

Research indicates that about 10 percent of patients who undergo bariatric surgery have unsatisfactory weight loss or regain much of the lost weight. Some behaviors, such as frequent snacking on high-calorie foods and lack of exercise, can contribute to inadequate weight loss. Technical problems that may occur with the operation, like a stretched pouch or separated stitches, may also contribute to inadequate weight loss.

Gastric Bypass Roux-en-Y (GBRY)

According to the American Society for Bariatric Surgery and the National Institutes of Health, Roux-en-Y gastric bypass is the most frequently performed weight-loss surgery in the United States. In this procedure, stapling creates a small (15 to 20 cc) stomach pouch. The remainder of the stomach is stapled completely closed and divided from the small pouch, but not removed. The outlet from the newly formed small pouch empties directly into the lower portion of the jejunum, thereby bypassing calorie absorption in the duodenum. The small intestine is divided just beyond the duodenum and one portion is used to construct a connection with the newly formed stomach pouch; the other end is connected into the side of the Roux limb of the intestine creating the “Y” shape that gives the technique its name. The length of either segment of the intestine can be increased to produce lower or higher levels of malabsorption. Advantages of GBRY include: the average excess weight loss is generally higher in a compliant patient than with purely restrictive procedures; one year after surgery weight loss averages about 75% of excess body weight; after 10 to 14 years 50-60% of excess body weight loss has been maintained in a significant subsets of patients; and roughly 96% of various associated health conditions (e.g., back pain, sleep apnea, high blood pressure, diabetes, and depression) are improved or resolved.

Risks associated with the Roux-en-Y gastric bypass procedure include: lowering of total-body iron and a predisposition to iron deficiency anemia; increased risk of osteoporosis; development of metabolic bone disease, resulting in bone pain, height loss, humped back, and fractures of the ribs and hips; chronic anemia due to Vitamin B12 deficiency; “dumping syndrome” (rapid emptying of stomach contents into the small intestine); stomach pouch stretching; and the bypassed portion of the stomach, duodenum and segments of the small intestine cannot be easily visualized using X-ray or endoscopy such that local problems (e.g., ulcers, bleeding or malignancy) can be difficult to detect and/or treat.

Extended (Distal) Roux-en-Y Gastric Bypass (RYGBP-E)

RYGBP-E is an alternative means of achieving malabsorption by creating a stapled or divided small gastric pouch, leaving the remainder of the stomach in place. A long limb of the small intestine is attached to the stomach to divert the bile and pancreatic juices. This procedure carries with it fewer operative risks because it avoids removal of the lower ¾ of the stomach. Gastric pouch size and the length of the bypassed intestine determine the risks for ulcers, malnutrition and other side effects.

Biliopancreatic Diversion (BPD)

Biliopancreatic diversion (BPD) is a complex bariatric operation that includes removing the lower portion of the stomach and creating a gastric sleeve (GS) with the small pouch that remains. The small intestine is then divided with one end attached to the stomach pouch to create what is called an “alimentary limb,” through which material travels from the stomach. The bile and pancreatic juices move through the “biliopancreatic limb,” which is connected to the side of the intestine close to the end. The biliopancreatic limb supplies digestive juices into the section of the intestine now called the “common limb.” The surgeon is able to vary the length of the common limb to regulate the amount of absorption of protein, fat, and fat-soluble vitamins.

BPD produces significant weight loss. However, the mortality rate is higher than with other bariatric operations, and there are more associated long-term complications due to decreased absorption of calories, vitamins, and minerals.

BPD with a “Duodenal Switch”

This procedure is a variation of BPD in which stomach removal is restricted to the outer margin, leaving a sleeve of stomach with the pylorus and the beginning of the duodenum at its end. The duodenum, the first portion of the small intestine, is divided so that pancreatic and bile drainage is bypassed. The near end of the “alimentary limb” is then attached to the beginning of the duodenum, while the “common limb” is created in the same way as described above.

Advantages of BPD, with or without a duodenal switch include: high degree of patient satisfaction because they are able to eat larger meals than with a purely restrictive or standard Roux-en-Y gastric bypass procedure; high levels of excess weight loss due to high levels of malabsorption; excess weight loss of 74% at one year, 78% at two years, 81% at three years, 84% at four years, and 91% at five years can be achieved; and long-term maintenance of excess body weight loss can be achieved if the patient adapts and adheres to a straightforward dietary, nutritional supplement, exercise and behavioral regimen. However, the BPD procedures carry with them a significant amount of risk, including: the initial period of intestinal adaptation during which bowel movements can be liquid and frequent; abdominal bloating and malodorous stool and/or gas may occur; close lifelong monitoring for protein malnutrition, anemia, and bone disease is recommended; lifelong vitamin supplementation is required; an increased risk of gallstone formation and the potential need for removal of the gallbladder; and intestinal irritation and ulcers.

### Body-Contouring Procedures

The outcome of any weight-loss regimen may be complemented by any of a number of body-contouring procedures. Cosmetic surgeons can reshape almost any area of the body using these techniques, which include liposuction, various lifts and tucks (tummy, body, arm), lipolysis, and photomology. Via body-sculpting surgery patients can effectively eliminate excess fat and skin that are unresponsive to diet, exercise, medication, or weight-loss surgery.

Liposuction is the most popular method of body-contouring surgery because it allows a surgeon to target specific areas of the body. Fat cells are permanently removed from the area using a minimally-invasive procedure. There are several types of liposuction procedures available, all of which use a wand-like instrument called a cannula to remove unwanted fat. Tumescent liposuction involves the injection of a large amount of anesthetic into the area being treated; so-called “wet” and “super-wet” techniques are variations of this type of liposuction. In ultrasonic assisted liposuction (UAL), sound waves are used to liquefy the fat before it is removed. Power assisted liposuction (PAL) employs a motor-powered cannula, which allows the surgeon to use smaller movements and make the experience less uncomfortable for the patient.

Recently, laser-assisted lipolysis has gained considerable attention. In this procedure, long-wavelength laser energy is applied to a target area, thus raising the temperature of the adipocytes and eventually causing apoptosis of the undesired cells. This minimally-invasive procedure can be performed under local anesthesia. Compared to traditional liposuction, laser-assisted lipolysis patients can expect a faster recovery and minimal bruising. Additionally, the heating that assists in the destruction of the adipocytes can also lead to skin retraction in the treated area, thus producing a smoother outward appearance.

Despite the progress made in the last 35 years in the field of body-sculpting procedures, little advancement has been made in developing a successful treatment for the appearance of cellulite, the condition where the skin of the lower limbs, abdomen, or pelvic region appears dimpled. Cellulite, which is more common in women than in men, is caused by a number of factors, including hormones and genetics. Cellulite is often relatively or completely unresponsive to diet, exercise, weight-reduction surgery, and traditional body-contouring procedures, such as liposuction. However, a procedure utilizing a combination of laser energy, light, vacuum technology, and physical pressure (called “photomology”) may prove helpful in the reduction of the appearance of cellulite for some patients. This process reduces the size of adipocytes near the skin surface by liquefying them and releasing the contained lipids, thereby restoring enlarged cells to a smaller, more-spherical shape. The result is an outward appearance of smoother, more-taught skin.

### Exemplary Compositions

In one embodiment, the present invention relates to a pharmaceutical composition comprising diethylpropion, metformin, and orlistat, or pharmaceutically acceptable salts or solvates of any of them; and at least one pharmaceutically acceptable carrier or excipient.

In one embodiment, the present invention relates to a pharmaceutical composition consisting essentially of diethylpropion, metformin, and orlistat, or pharmaceutically acceptable salts or solvates of any of them; and at least one pharmaceutically acceptable carrier or excipient.

In one embodiment, the present invention relates to a pharmaceutical composition consisting of diethylpropion, metformin, and orlistat, or pharmaceutically acceptable salts or solvates of any of them; and at least one pharmaceutically acceptable carrier or excipient.

In one embodiment, the present invention relates to any one of the above-mentioned compositions, wherein the amount of diethylpropion is about 20 mg to about 90 mg.

In one embodiment, the present invention relates to any one of the above-mentioned compositions, wherein the amount of diethylpropion is about 20-30 mg, about 32.5-42.5 mg, about 40-60 mg, or about 60-90 mg.

In one embodiment, the present invention relates to any one of the above-mentioned compositions, wherein the amount of diethylpropion is about 25 mg, about 37.5 mg, about 50 mg, or about 75 mg.

In one embodiment, the present invention relates to any one of the above-mentioned compositions, wherein the amount of metformin is about 225 mg to about 2200 mg.

In one embodiment, the present invention relates to any one of the above-mentioned compositions, wherein the amount of metformin is about 225-275 mg, about 450-550 mg, about 700-800 mg, about 900-1100 mg, about 1350-1650 mg, or about 1800-2200 mg.

In one embodiment, the present invention relates to any one of the above-mentioned compositions, wherein the amount of metformin is about 250 mg, about 500 mg, about 750 mg, about 1000 mg, about 1500 mg, or about 2000 mg.

In one embodiment, the present invention relates to any one of the above-mentioned compositions, wherein the amount of orlistat is about 50 mg to about 390 mg.

In one embodiment, the present invention relates to any one of the above-mentioned compositions, wherein the amount of orlistat is about 50-70 mg, about 110-130 mg, about 220-260 mg, or about 330-390 mg.

In one embodiment, the present invention relates to any one of the above-mentioned compositions, wherein the amount of orlistat is about 60 mg, about 120 mg, about 240 mg, or about 360 mg.

In one embodiment, the present invention relates to any one of the above-mentioned pharmaceutical compositions, wherein the pharmaceutical composition is in the form of a tablet, pill, capsule, or elixir.

### Exemplary Methods

In one embodiment, the present invention relates to a method of treating obesity, comprising the step of co-administering to a subject in need thereof a therapeutically effective amount of diethylpropion, metformin, and orlistat, or pharmaceutically acceptable salts or solvates of any of them.

In one embodiment, the present invention relates to a method of achieving weight loss, comprising the step of co-administering to a subject in need thereof a therapeutically effective amount of diethylpropion, metformin, and orlistat, or pharmaceutically acceptable salts or solvates of any of them.

In one embodiment, the present invention relates to any one of the above-mentioned methods, wherein diethylpropion is administered once daily. In one embodiment, the present invention relates to any one of the above-mentioned methods, wherein diethylpropion is administered twice daily.

In one embodiment, the present invention relates to any one of the above-mentioned methods, wherein metformin is administered once daily. In one embodiment, the present invention relates to any one of the above-mentioned methods, wherein metformin is administered twice daily.

In one embodiment, the present invention relates to any one of the above-mentioned methods, wherein orlistat is administered once daily. In one embodiment, the present invention relates to any one of the above-mentioned methods, wherein orlistat is administered twice daily. In one embodiment, the present invention relates to any one of the above-mentioned methods, wherein orlistat is administered three times daily.

In one embodiment, the present invention relates to any one of the above-mentioned methods, wherein about 20 mg to about 90 mg of diethylpropion is administered.

In one embodiment, the present invention relates to any one of the above-mentioned methods, wherein about 20-30 mg, about 32.5-42.5 mg, about 40-60 mg, or about 60-90 mg of diethylpropion is administered.

In one embodiment, the present invention relates to any one of the above-mentioned methods, wherein about 25 mg, about 37.5 mg, about 50 mg, or about 75 mg of diethylpropion is administered.

In one embodiment, the present invention relates to any one of the above-mentioned methods, wherein about 225 mg to about 2200 mg of metformin is administered.

In one embodiment, the present invention relates to any one of the above-mentioned methods, wherein about 225-275 mg, about 450-550 mg, about 700-800 mg, about 900-1100 mg, about 1350-1650 mg, or about 1800-2200 mg of metformin is administered.

In one embodiment, the present invention relates to any one of the above-mentioned methods, wherein about 250 mg, about 500 mg, about 750 mg, about 1000 mg, about 1500 mg, or about 2000 mg of metformin is administered.

In one embodiment, the present invention relates to any one of the above-mentioned methods, wherein about 50 mg to about 390 mg of orlistat is administered.

In one embodiment, the present invention relates to any one of the above-mentioned methods, wherein about 50-70 mg, about 110-130 mg, about 220-260 mg, or about 330-390 mg of orlistat is administered.

In one embodiment, the present invention relates to any one of the above-mentioned methods, wherein about 60 mg, about 120 mg, about 240 mg, or about 360 mg of orlistat is administered.

In one embodiment, the present invention relates to any one of the above-mentioned methods, wherein a dose of diethylpropion is administered once daily; and the dose is about 37.5 mg of diethylpropion. In one embodiment, the present invention relates to any one of the above-mentioned methods, wherein a dose of diethylpropion is administered twice daily; and each dose is about 37.5 mg of diethylpropion. In one embodiment, the present invention relates to any one of the above-mentioned methods, wherein a dose of diethylpropion is administered once daily; and the dose is about 25 mg of diethylpropion. In one embodiment, the present invention relates to any one of the above-mentioned methods, wherein a dose of diethylpropion is administered twice daily; and each dose is about 25 mg of diethylpropion.

In one embodiment, the present invention relates to any one of the above-mentioned methods, wherein a dose of metformin is administered once daily; and the dose is about 1000 mg of metformin. In one embodiment, the present invention relates to any one of the above-mentioned methods, wherein a dose of metformin is administered twice daily; and each dose is about 500 mg of metformin. In one embodiment, the present invention relates to any one of the above-mentioned methods, wherein a dose of metformin is administered once daily; and the dose is about 500 mg of metformin. In one embodiment, the present invention relates to any one of the above-mentioned methods, wherein a dose of metformin is administered twice daily; and each dose is about 750 mg of metformin.

In one embodiment, the present invention relates to any one of the above-mentioned methods, wherein a dose of orlistat is administered once daily; and the dose is about 120 mg of orlistat. In one embodiment, the present invention relates to any one of the above-mentioned methods, wherein a dose of orlistat is administered twice daily; and each dose is about 120 mg of orlistat. In one embodiment, the present invention relates to any one of the above-mentioned methods, wherein a dose of orlistat is administered three times daily; and each dose is about 120 mg of orlistat.

In one embodiment, the present invention relates to any one of the above-mentioned methods, wherein

a dose of diethylpropion is administered once daily; the dose of diethylpropion is about 37.5 mg of diethylpropion;
a dose of metformin is administered once daily; the dose of metformin is about 500 mg of metformin;
a dose of orlistat is administered twice daily; and each dose of orlistat is about 120 mg of orlistat.

In one embodiment, the present invention relates to any one of the above-mentioned methods, wherein the dose of diethylpropion, the dose of metformin, or the dose of orlistat is adjusted as needed to treat obesity or to achieve weight loss in said subject.

In one embodiment, the present invention relates to any one of the above-mentioned methods, wherein the above-mentioned method is used for a period of treatment. In one embodiment, the present invention relates to any one of the above-mentioned methods, wherein the period of treatment is about 1 week to about 36 months. In one embodiment, the present invention relates to any one of the above-mentioned methods, wherein the period of treatment is about 1, 2, 3, 4, 5, 6, 7, or 8 weeks. In one embodiment, the present invention relates to any one of the above-mentioned methods, wherein the period of treatment is about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months.

In one embodiment, the present invention relates to any one of the above-mentioned methods, further comprising the step of treating the subject with a medical device.

In one embodiment, the present invention relates to any one of the above-mentioned methods, further comprising the step of treating the subject with a medical device, wherein the medical device is selected from the group consisting of an endoluminal sleeve, an intragastric balloon, a fastener, a gastric pacemaker, and an electrical device.

In one embodiment, the present invention relates to any one of the above-mentioned methods, further comprising the step of treating the subject with a medical device, wherein the medical device is an endoluminal sleeve.

In one embodiment, the present invention relates to any one of the above-mentioned methods, further comprising the step of treating the subject with a medical device, wherein the medical device is a fastener.

In one embodiment, the present invention relates to any one of the above-mentioned methods, further comprising the step of treating the subject with a medical device, wherein the medical device is a fastener; and the fastener comprises anchors, staples, blind staples, bands, clips, tags, adhesives, or screws.

In one embodiment, the present invention relates to any one of the above-mentioned methods, further comprising the step of treating the subject with a medical device, wherein the medical device is a gastric pacemaker.

In one embodiment, the present invention relates to any one of the above-mentioned methods, further comprising the step of treating the subject with a medical device, wherein the medical device is an electrical device.

In one embodiment, the present invention relates to any one of the above-mentioned methods, further comprising the step of treating the subject with a medical device, wherein the medical device is an electrical device; and the electrical device is used to stimulate the vagus nerve of the subject.

In one embodiment, the present invention relates to any one of the above-mentioned methods, further comprising the step of treating the subject with a medical device, wherein the medical device is an electrical device; and the electrical device is used to stimulate the brain of the subject.

In one embodiment, the present invention relates to any one of the above-mentioned methods, further comprising the step of treating the subject with a medical device, wherein the medical device is an electrical device; and the electrical device is used to stimulate the metabolism of the subject.

In one embodiment, the present invention relates to any one of the above-mentioned methods, further comprising the step of treating the subject with a medical device, wherein the medical device is implanted before initiation of co-administration of diethylpropion, metformin, and orlistat. In one embodiment, the present invention relates to any one of the above-mentioned methods, further comprising the step of treating the subject with a medical device, wherein co-administration of diethylpropion, metformin, and orlistat is initiated after implantation of the medical device, thereby preventing or decreasing weight re-gain or weight-loss plateau after implantation of the device.

In one embodiment, the present invention relates to any one of the above-mentioned methods, further comprising the step of treating the subject with a medical device, wherein co-administration of diethylpropion, metformin, and orlistat is initiated before implantation of the medical device. In one embodiment, the present invention relates to any one of the above-mentioned methods, further comprising the step of treating the subject with a medical device, wherein co-administration of diethylpropion, metformin, and orlistat is initiated before implantation of the medical device, thereby preventing or decreasing weight re-gain or weight-loss plateau after implantation of the device.

In one embodiment, the present invention relates to any one of the above-mentioned methods, further comprising the step of treating the subject with a medical device, wherein co-administration of diethylpropion, metformin, and orlistat is initiated approximately simultaneously with implantation of the medical device. In one embodiment, the present invention relates to any one of the above-mentioned methods, further comprising the step of treating the subject with a medical device, wherein co-administration of diethylpropion, metformin, and orlistat is initiated approximately simultaneously with implantation of the medical device, thereby preventing or decreasing weight re-gain or weight-loss plateau after implantation of the device.

In one embodiment, the present invention relates to any one of the above-mentioned methods, further comprising the step of completing a medical procedure on the subject.

In one embodiment, the present invention relates to any one of the above-mentioned methods, further comprising the step of completing a medical procedure on the subject; wherein the medical procedure is a restrictive procedure or a malabsorptive procedure.

In one embodiment, the present invention relates to any one of the above-mentioned methods, further comprising the step of completing a medical procedure on the subject; wherein the medical procedure is a restrictive procedure.

In one embodiment, the present invention relates to any one of the above-mentioned methods, further comprising the step of completing a medical procedure on the subject; wherein the medical procedure is a restrictive procedure; and the restrictive procedure is selected from the group consisting of adjustable gastric band surgery, vertical banded gastroplasty, and gastric sleeve surgery.

In one embodiment, the present invention relates to any one of the above-mentioned methods, further comprising the step of completing a medical procedure on the subject; wherein the medical procedure is a restrictive procedure; and the restrictive procedure is adjustable gastric band surgery.

In one embodiment, the present invention relates to any one of the above-mentioned methods, further comprising the step of completing a medical procedure on the subject; wherein the medical procedure is a malabsorptive procedure.

In one embodiment, the present invention relates to any one of the above-mentioned methods, further comprising the step of completing a medical procedure on the subject; wherein the medical procedure is a malabsorptive procedure; and the malabsorptive procedure is selected from the group consisting of gastric bypass Roux-en-Y, extended Roux-en-Y gastric bypass, biliopancreatic diversion, and biliopancreatic diversion with a duodenal switch.

In one embodiment, the present invention relates to any one of the above-mentioned methods, further comprising the step of completing a medical procedure on the subject; wherein the medical procedure is a malabsorptive procedure; and the malabsorptive procedure is gastric bypass Roux-en-Y.

In one embodiment, the present invention relates to any one of the above-mentioned methods, further comprising the step of completing a medical procedure on the subject; wherein the medical procedure is a body-contouring procedure.

In one embodiment, the present invention relates to any one of the above-mentioned methods, further comprising the step of completing a medical procedure on the subject; wherein the medical procedure is a body-contouring procedure; and the body-contouring procedure is selected from the group consisting of liposuction, a tuck, a lift, lipolysis, and photomology.

In one embodiment, the present invention relates to any one of the above-mentioned methods, further comprising the step of completing a medical procedure on the subject; wherein the medical procedure is a body-contouring procedure; and the body-contouring procedure is selected from the group consisting of liposuction, lipolysis, and photomology.

In one embodiment, the present invention relates to any one of the above-mentioned methods, further comprising the step of completing a medical procedure on the subject; wherein the medical procedure is a body-contouring procedure; and the body-contouring procedure is liposuction.

In one embodiment, the present invention relates to any one of the above-mentioned methods, further comprising the step of completing a medical procedure on the subject; wherein the medical procedure is a body-contouring procedure; and the body-contouring procedure is lipolysis.

In one embodiment, the present invention relates to any one of the above-mentioned methods, further comprising the step of completing a medical procedure on the subject; wherein the medical procedure is a body-contouring procedure; the body-contouring procedure is lipolysis; and the lipolysis is laser-assisted lipolysis.

In one embodiment, the present invention relates to any one of the above-mentioned methods, further comprising the step of completing a medical procedure on the subject; wherein the medical procedure is a body-contouring procedure; and the body-contouring procedure is photomology.

In one embodiment, the present invention relates to any one of the above-mentioned methods, further comprising the step of completing a medical procedure on the subject, wherein the medical procedure is completed before initiation of co-administration of diethylpropion, metformin, and orlistat. In one embodiment, the present invention relates to any one of the above-mentioned methods, further comprising the step of completing a medical procedure on the subject, wherein co-administration of diethylpropion, metformin, and orlistat is initiated after completion of the medical procedure, thereby preventing or decreasing weight re-gain or weight-loss plateau after completion of the procedure.

In one embodiment, the present invention relates to any one of the above-mentioned methods, further comprising the step of completing a medical procedure on the subject, wherein co-administration of diethylpropion, metformin, and orlistat is initiated before completion of the medical procedure. In one embodiment, the present invention relates to any one of the above-mentioned methods, further comprising the step of completing a medical procedure on the subject, wherein co-administration of diethylpropion, metformin, and orlistat is initiated before completion of the medical procedure, thereby preventing or decreasing weight re-gain or weight-loss plateau after completion of the procedure.

In one embodiment, the present invention relates to any one of the above-mentioned methods, further comprising the step of completing a medical procedure on the subject, wherein co-administration of diethylpropion, metformin, and orlistat is initiated approximately simultaneously with completion of the medical procedure. In one embodiment, the present invention relates to any one of the above-mentioned methods, further comprising the step of completing a medical procedure on the subject, wherein co-administration of diethylpropion, metformin, and orlistat is initiated approximately simultaneously with completion of the medical procedure, thereby preventing or decreasing weight re-gain or weight-loss plateau after completion of the medical procedure.

In one embodiment, the present invention relates to any one of the above-mentioned methods, further comprising the step of completing a second medical procedure on the subject.

In addition to achieving weight loss or treating obesity, any one of the above-mentioned methods can be used in a subject in need thereof, alone or in combination with other forms of treatment, to treat a malady selected from the group consisting of type 2 diabetes, shortness of breath, gallbladder disease, hypertension, elevated blood cholesterol levels, cancer (e.g., endometrial, breast, prostate, colon), osteoarthritis, other orthopedic problems, reflux esophagitis (heartburn), snoring, sleep apnea, menstrual irregularities, infertility, heart trouble, dyslipidemia, coronary heart disease, stroke, hyperinsulinemia, depression, anxiety, gout, fatty liver disease, and insulin resistance. In certain embodiments, the malady is type 2 diabetes, hyperinsulinemia, or insulin resistance. In certain embodiments, the malady is type 2 diabetes.

### EXEMPLIFICATION

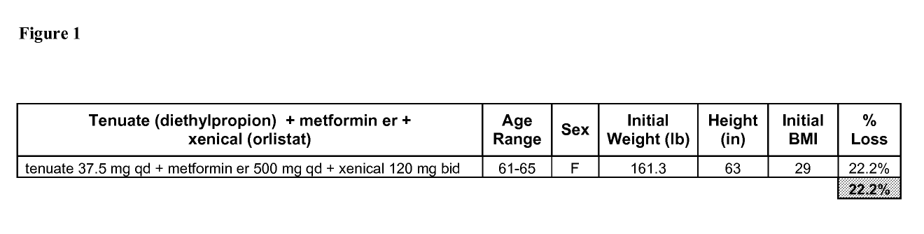

The example below describes a specific ternary combination therapy for the treatment of obesity. Specific dosage amounts and regimens are tabulated in FIG. 1. In the Figure, “qd” is used to mean “once daily,” “bid” is used to mean “twice daily,” and “tid” is used to mean “three times daily.” It should be noted that the “Initial Weight” of the subjects was taken before any treatment was administered. Because the effectiveness of the dosage amounts and regimens differs slightly for each patient, treatment plans were adjusted over the course of treatment based on results obtained. The treatment plan listed for each patient was the specific combination that was deemed most effective for that patient, not necessarily the exact treatment plan that was administered for the entire treatment period.

### Example 1

FIG. 1 tabulates the data collected from a patient administered a combination of diethylpropion, metformin, and orlistat. The patient was in the 61-65 age range and had an initial BMI of 29. This patient lost 22.2% of her initial body weight.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein.

I claim:

1. A pharmaceutical composition comprising diethylpropion, metformin, and orlistat, or pharmaceutically acceptable salts or solvates of any of them; and at least one pharmaceutically acceptable carrier or excipient.

2. The composition of claim 1, wherein the amount of diethylpropion is about 20 mg to about 90 mg.

3. The composition of claim 1, wherein the amount of diethylpropion is about 20-30 mg, about 32.5-42.5 mg, about 40-60 mg, or about 60-90 mg.

4. The composition of claim 1, wherein the amount of diethylpropion is about 25 mg, about 37.5 mg, about 50 mg, or about 75 mg.

5. The composition of claim 1, wherein the amount of metformin is about 225 mg to about 2200 mg.

6. The composition of claim 1, wherein the amount of metformin is about 225-275 mg, about 450-550 mg, about 700-800 mg, about 900-1100 mg, about 1350-1650 mg, or about 1800-2200 mg.

7. The composition of claim 1, wherein the amount of metformin is about 250 mg, about 500 mg, about 750 mg, about 1000 mg, about 1500 mg, or about 2000 mg.

8. The composition of claim 1, wherein the amount of orlistat is about 50 mg to about 390 mg.

9. The composition of claim 1, wherein the amount of orlistat is about 50-70 mg, about 110-130 mg, about 220-260 mg, or about 330-390 mg.

10. The composition of claim 1, wherein the amount of orlistat is about 60 mg, about 120 mg, about 240 mg, or about 390 mg.

11. A method of treating obesity or achieving weight loss, comprising the step of co-administering to a subject in need thereof a therapeutically effective amount of diethylpropion, metformin, and orlistat, or pharmaceutically acceptable salts or solvates of any of them.

12. The method of claim 11, wherein diethylpropion is administered once daily.

13. The method of claim 11, wherein metformin is administered once daily.

14. The method of claim 11, wherein orlistat is administered twice daily.

15. The method of claim 11, wherein about 20 mg to about 90 mg of diethylpropion is administered.

16. The method of claim 11, wherein about 20-30 mg, about 32.5-42.5 mg, about 40-60 mg, or about 60-90 mg of diethylpropion is administered.

17. The method of claim 11, wherein about 25 mg, about 37.5 mg, about 50 mg, or about 75 mg of diethylpropion is administered.

18. The method of claim 11, wherein about 225 mg to about 2200 mg of metformin is administered.

19. The method of claim 11, wherein about 225-275 mg, about 450-550 mg, about 700-800 mg, about 900-1100 mg, about 1350-1650 mg, or about 1800-2200 mg of metformin is administered.

20. The method of claim 11, wherein about 250 mg, about 500 mg, about 750 mg, about 1000 mg, about 1500 mg, or about 2000 mg of metformin is administered.

21. The method of claim 11, wherein about 50 mg to about 390 mg of orlistat is administered.

22. The method of claim 11, wherein about 50-70 mg, about 110-130 mg, about 220-260 mg, or about 330-390 mg of orlistat is administered.

23. The method of claim 11, wherein about 60 mg, about 120 mg, about 240 mg, or about 360 mg of orlistat is administered.

24. The method of claim 11, wherein
a dose of diethylpropion is administered once daily; the dose of diethylpropion is about 37.5 mg of diethylpropion;
a dose of metformin is administered once daily; the dose of metformin is about 500 mg of metformin;
a dose of orlistat is administered twice daily; and each dose of orlistat is about 120 mg of orlistat.

25. The method of claim 11, further comprising the step of treating the subject with a medical device.

26. The method of claim 11, further comprising the step of treating the subject with a medical device, wherein the medical device is selected from the group consisting of an endoluminal sleeve, an intragastric balloon, a fastener, a gastric pacemaker, and an electrical device.

27. The method of claim 11, further comprising the step of completing a medical procedure on the subject.

28. The method of claim 11, further comprising the step of completing a medical procedure on the subject; wherein the medical procedure is a restrictive procedure, a malabsorptive procedure, or a body-contouring procedure.

29. A method of treating obesity or achieving weight loss, comprising the step of administering to a subject in need thereof a therapeutically effective amount of a pharmaceutical composition of claim 1.

30. A method of treating a malady, comprising the step of co-administering to a subject in need thereof a therapeutically effective amount of diethylpropion, metformin, and orlistat, or pharmaceutically acceptable salts or solvates of any of them, wherein the malady is selected from the group consisting of type 2 diabetes, shortness of breath, gallbladder disease, hypertension, elevated blood cholesterol levels, cancer (e.g., endometrial, breast, prostate, colon), osteoarthritis, other orthopedic problems, reflux esophagitis (heartburn), snoring, sleep apnea, menstrual irregularities, infertility, heart trouble, dyslipidemia, coronary heart disease, stroke, hyperinsulinemia, depression, anxiety, gout, fatty liver disease, and insulin resistance.
